(19) United States
(12) Patent Application Publication
(10) Pub. No.: US 2019/0269334 A1
(43) Pub. Date: Sep. 5, 2019

(54) PHYSIOLOGICAL PARAMETER MONITORING

(71) Applicant: Covidien LP, Mansfield, MA (US)

(72) Inventors: Paul S. Addison, Edinburgh (GB); Dean Montgomery, Edinburgh (GB); Andre Antunes, Edinburgh (GB)

(21) Appl. No.: 15/911,449

(22) Filed: Mar. 5, 2018

(51) Int. Cl. A61B 5/021; A61B 5/00; A61B 5/04; G06F 17/11

(52) U.S. Cl. CPC A61B 5/021; A61B 5/7271; G06F 17/11; A61B 5/04; A61B 5/742

(57) ABSTRACT

In some examples, a device includes a display and processing circuitry configured to receive a physiological signal indicative of a physiological parameter of a patient. The processing circuitry is also configured to determine a first set of mean arterial pressure values of the patient for a time period based on the physiological signal using a first algorithm. In some examples, the processing circuitry is further configured to determine an autoregulation status of the patient based on the first set of mean arterial pressure values. In some examples, the processing circuitry is configured to present, via the display, a second set of mean arterial pressure values of the patient for the time period, wherein the second set of mean arterial pressure values are determined using a second algorithm different from the first algorithm.

### TECHNICAL FIELD

This disclosure relates to physiological parameter monitoring.

### BACKGROUND

Cerebral autoregulation (CA) is the response mechanism by which an organism regulates cerebral blood flow over a wide range of systemic blood pressure changes through complex myogenic, neurogenic, and metabolic mechanisms. Autoregulation dysfunction may result from a number of causes including, stroke, traumatic brain injury, brain lesions, brain asphyxia, or infections of the central nervous system. Intact cerebral autoregulation function occurs over a range of blood pressures defined between a lower limit of autoregulation (LLA) and an upper limit of autoregulation (ULA).

### SUMMARY

This disclosure describes devices, systems, and techniques for determining at least two sets of mean arterial pressure (MAP) values based on a sensed physiological signal of a patient and presenting meaningful information regarding the MAP values to a user. In some examples, a regional oximetry device is configured to determine an autoregulation status of a patient based on a first set of MAP values of the patient. The regional oximetry device may also be configured to present, via a display, a second set of MAP values of the patient. The first and second sets of MAP values of the patient may be representative of the MAP of the patient for the same time period, but may differ from each other, e.g., because the first and second sets of MAP values were determined using different MAP calculation algorithms. In some cases, the first and second sets of MAP values are determined using the same segment of a sensed physiological signal.

In some examples, the regional oximetry device may determine the first set of MAP values and the second set of MAP values and communicate the second set of MAP values to a multi-parametric monitor (MPM) device. In another example, the regional oximetry device may receive the first set of MAP values and/or the second set of MAP values from the MPM device. In a further example, the regional oximetry device may determine the second set of MAP values to mimic the determination of MAP by the MPM device.

Clause 1: In some examples, a device comprises a display and processing circuitry configured to receive a physiological signal indicative of a physiological parameter of a patient and determine a first set of mean arterial pressure values of the patient for a time period based on the physiological signal using a first algorithm. The processing circuitry is also configured to determine an autoregulation status of the patient based on the first set of mean arterial pressure values. The processing circuitry is further configured to present, via the display, a second set of mean arterial pressure values of the patient for the time period, wherein the second set of mean arterial pressure values are determined using a second algorithm different from the first algorithm.

Clause 2: In some examples of clause 1, the processing circuitry is further configured to determine the second set of mean arterial pressure values based on the physiological signal using the second algorithm and communicate the second set of mean arterial pressure values to a multi-parametric monitor device.

Clause 3: In some examples of clause 1 or clause 2, the processing circuitry is further configured to determine the second set of mean arterial pressure values based on the physiological signal using the second algorithm to mimic determination of mean arterial pressure for the time period by a multi-parametric monitor device.

Clause 4: In some examples of clause 3, the processing circuitry is configured to determine the second set of values for mean arterial pressure using the second algorithm to mimic determination of mean arterial pressure by at least determining a weighted average of a systolic peak of the physiological signal and a diastolic peak of the physiological signal. A weighting of the diastolic peak of the physiological signal is greater than or equal to a weighting of the systolic peak of the physiological signal.

Clause 5: In some examples of any of clauses 1-4, the processing circuitry is further configured to receive the second set of mean arterial pressure values from a multi-parametric monitor device.

Clause 6: In some examples of any of clauses 1-5, the physiological parameter of the patient comprises a first physiological parameter, and the processing circuitry is further configured to receive a second physiological signal of the patient, the second physiological signal being indicative of a second physiological parameter different from the first physiological parameter. The processing circuitry is also configured to determine a set of oxygen saturation values of the patient based on the second physiological signal and determine a relative time delay between the first set of mean arterial pressure values and the set of oxygen saturation values. The processing circuitry is configured to determine the autoregulation status of the patient based on the first set of mean arterial pressure values, the set of oxygen saturation values, and the relative time delay between the first set of mean arterial pressure values and the set of oxygen saturation values.

Clause 7: In some examples of clause 6, the processing circuitry is configured to determine the first set of mean arterial pressure values based on a sampling window for the first set of mean arterial pressure values. The processing circuitry is configured to determine the set of oxygen saturation values based on a sampling window for the set of oxygen saturation values. The processing circuitry is configured to determine the relative time delay between the first set of mean arterial pressure values and the set of oxygen saturation values based on a duration of the sampling window for the first set of mean arterial pressure values and a duration of the sampling window for the set of oxygen saturation values.

Clause 8: In some examples of clause 6 or clause 7, the processing circuitry is configured to determine the relative time delay between the first set of mean arterial pressure values and the set of oxygen saturation values by at least determining a cross-correlation function for the first physiological signal and the second physiological signal and determining a time lag at which the cross-correlation function has a maximum amplitude.

Clause 9: In some examples of any of clauses 1-8, the processing circuitry is further configured to synchronize the first set of mean arterial pressure values and the set of oxygen saturation values based on the relative time delay. The processing circuitry is configured to determine the autoregulation status of the patient based on the synchronized first set of mean arterial pressure values and the synchronized set of oxygen saturation values.

Clause 10: In some examples of any of clauses 1-9, the processing circuitry is further configured to determine that the autoregulation status is impaired based on the first set of mean arterial pressure values and a set of oxygen saturation values of the patient and generate a notification in response to determining that the autoregulation status is impaired.

Clause 11: In some examples of any of clauses 1-10, the processing circuitry is further configured to determine a signal quality metric based on the difference between the first set of mean arterial pressure values and the second set of mean arterial pressure values. The processing circuitry is also configured to determine that the signal quality metric is greater than or equal to a threshold level and generate a notification in response to determining that the signal quality metric is greater than or equal to the threshold level. The processing circuitry is configured to output the autoregulation status before determining that the signal quality metric is greater than or equal to the threshold level and refrain from further outputting the autoregulation status in response to determining that the signal quality metric is greater than or equal to the threshold level.

Clause 12: In some examples, a method comprises receiving, by processing circuitry, a physiological signal indicative of a physiological parameter of a patient. The method also comprises determining, by the processing circuitry, a first set of mean arterial pressure values of the patient for a time period based on the physiological signal using a first algorithm. The method further comprises determining, by the processing circuitry, an autoregulation status of the patient based on the first set of mean arterial pressure values. The method also comprises presenting, via a display, a second set of mean arterial pressure values of the patient for the time period, wherein the second set of mean arterial pressure values are determined using a second algorithm different from the first algorithm.

Clause 13: In some examples of clause 12, the method further comprises determining the second set of mean arterial pressure values based on the physiological signal using the second algorithm and communicating the second set of mean arterial pressure values to a multi-parametric monitor device.

Clause 14: In some examples of clause 12 or clause 13, the method further comprises receiving the second set of mean arterial pressure values from a multi-parametric monitor device.

Clause 15: In some examples of any of clauses 12-14, the method further comprises receiving, from the sensing circuitry, a second physiological signal of the patient, the second physiological signal being indicative of a second physiological parameter different from the first physiological parameter. The method also comprises determining a set of oxygen saturation values of the patient based on the second physiological signal. The method further comprises determining a relative time delay between the first set of mean arterial pressure values and the set of oxygen saturation values. Determining the autoregulation status of the patient is based on the first set of mean arterial pressure values, the set of oxygen saturation values, and the relative time delay between the first set of mean arterial pressure values and the set of oxygen saturation values.

Clause 16: In some examples of clause 15, determining the first set of mean arterial pressure values is further based on a sampling window for the first set of mean arterial pressure values. Determining the set of oxygen saturation values is further based on a sampling window for the set of oxygen saturation values. Determining the relative time delay between the first set of mean arterial pressure values and the set of oxygen saturation values based on a duration of the sampling window for the first physiological signal and a duration of the sampling window for the second physiological signal.

Clause 17: In some examples of clause 15 or clause 16, determining the relative time delay between the first set of mean arterial pressure values and the set of oxygen saturation values comprises determining a cross-correlation function for the first physiological signal and the second physiological signal and determining a time lag at which the cross-correlation function has a maximum amplitude.

Clause 18: In some examples, a device comprises a display and processing circuitry configured to receive a first physiological signal indicative of a physiological parameter of a patient and determine a first set of mean arterial pressure values of the patient for a time period based on the first physiological signal. The processing circuitry is also configured to transmit the first set of mean arterial pressure values to a multi-parametric monitor device and determine an autoregulation status of the patient based on the first set of mean arterial pressure values. The processing circuitry is further configured to present, via the display, the first set of mean arterial pressure values of the patient for the time period.

Clause 19: In some examples of clause 18, the physiological parameter of the patient comprises a first physiological parameter, and the processing circuitry is further configured to receive a second physiological signal of the patient, the second physiological signal being indicative of a second physiological parameter different from the first physiological parameter. The processing circuitry is also configured to determine a set of oxygen saturation values of the patient based on the second physiological signal and determine a relative time delay between the first set of mean arterial pressure values and the set of oxygen saturation values. The processing circuitry is configured to determine the autoregulation status of the patient based on the first set of mean arterial pressure values, the set of oxygen saturation values, and the relative time delay between the first set of mean arterial pressure values and the set of oxygen saturation values.

Clause 20: In some examples of clause 18 or clause 19, the processing circuitry is configured to determine the first set of mean arterial pressure values based on a sampling window for the first set of mean arterial pressure values. The processing circuitry is configured to determine the set of oxygen saturation values based on a sampling window for the set of oxygen saturation values. The processing circuitry is configured to determine the relative time delay between the first set of mean arterial pressure values and the set of oxygen saturation values based on a duration of the sampling window for the first set of mean arterial pressure values and a duration of the sampling window for the set of oxygen saturation values.

Clause 21: In some examples, a system comprises means for receiving a physiological signal indicative of a physiological parameter of a patient; means for determining a first set of mean arterial pressure values of the patient for a time period based on the physiological signal using a first algorithm; means for determining an autoregulation status of the patient based on the first set of mean arterial pressure values; and means for presenting a second set of mean arterial pressure values of the patient for the time period, wherein the second set of mean arterial pressure values are determined using a second algorithm different from the first algorithm.

Clause 22: In some examples, a device comprises a display and processing circuitry configured to receive a first physiological signal of a patient and a second physiological signal of the patient, the first and second physiological signals being indicative of different physiological parameters of the patient. The processing circuitry is also configured to determine a set of mean arterial pressure values of the patient based on the first physiological signal and determine a set of oxygen saturation values of the patient based on the second physiological signal. The processing circuitry is further configured to determine a relative time delay between the set of mean arterial pressure values and the set of oxygen saturation values and determine an autoregulation status of the patient based on the set of mean arterial pressure values, the set of oxygen saturation values, and the relative time delay between the set of mean arterial pressure values and the set of oxygen saturation values. The processing circuitry is configured to present, via the display, an indication of the autoregulation status.

Clause 23: In some examples of clause 22, the processing circuitry is configured to determine the set of mean arterial pressure values based on a sampling window for the set of mean arterial pressure values. The processing circuitry is also configured to determine the set of oxygen saturation values based on a sampling window for the set of oxygen saturation values. The processing circuitry is configured to determine the relative time delay between the set of mean arterial pressure values and the set of oxygen saturation values based on a duration of the sampling window for the set of mean arterial pressure values and a duration of the sampling window for the set of oxygen saturation values.

Clause 24: In some examples of clause 23, the processing circuitry is configured to determine the relative time delay between the set of mean arterial pressure values and the set of oxygen saturation values by at least determining a cross-correlation function for the first physiological signal and the second physiological signal; and determining a time lag at which the cross-correlation function has a maximum amplitude.

Clause 25: In some examples of any of clauses 22-24, the processing circuitry is further configured to synchronize the set of mean arterial pressure values and the set of oxygen saturation values based on the relative time delay. The processing circuitry is also configured to determine the autoregulation status of the patient based on the synchronized set of mean arterial pressure values and the synchronized set of oxygen saturation values.

Clause 26: In some examples, a method comprises receiving, by processing circuitry, a first physiological signal of a patient and a second physiological signal of the patient, the first and second physiological signals being indicative of different physiological parameters of the patient. The method further comprises determining, by the processing circuitry, a set of mean arterial pressure values of the patient based on the first physiological signal and determine a set of oxygen saturation values of the patient based on the second physiological signal; determining, by the processing circuitry, a relative time delay between the set of mean arterial pressure values and the set of oxygen saturation values and determine an autoregulation status of the patient based on the set of mean arterial pressure values, the set of oxygen saturation values, and the relative time delay between the set of mean arterial pressure values and the set of oxygen saturation values. The method may further comprise presenting, via a display, an indication of the autoregulation status.

Clause 27: In some examples, a system comprises means for receiving a first physiological signal of a patient and a second physiological signal of the patient, the first and second physiological signals being indicative of different physiological parameters of the patient. The system further comprises means for determining a set of mean arterial pressure values of the patient based on the first physiological signal and determine a set of oxygen saturation values of the patient based on the second physiological signal; means for determining a relative time delay between the set of mean arterial pressure values and the set of oxygen saturation values and determine an autoregulation status of the patient based on the set of mean arterial pressure values, the set of oxygen saturation values, and the relative time delay between the set of mean arterial pressure values and the set of oxygen saturation values. The system may further comprise means for presenting an indication of the autoregulation status.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual block diagram illustrating an example regional oximetry device and an example multi-parametric (MPM) device.

FIG. 2 is a conceptual block diagram illustrating an example regional oximetry device configured to monitor the autoregulation status of a patient.

FIG. 3 illustrates an example graphical user interface including autoregulation information presented on a display.

FIG. 4 is a conceptual block diagram illustrating an example physiological parameter monitoring system.

FIG. 5 is a perspective-view diagram illustrating an example physiological parameter monitoring system.

FIGS. 6A and 6B illustrate example blood-pressure waveforms.

FIG. 7 is a flow diagram illustrating example techniques for presenting mean arterial pressure (MAP) values.

FIGS. 8A and 8B are a flow diagram and a conceptual block diagram, respectively, illustrating an example technique performed by a regional oximetry device to determine and display MAP to mimic the MAP determined by an MPM device, as well as a corresponding example technique performed by the MPM device.

FIGS. 9A and 9B are a flow diagram and a conceptual block diagram illustrating an example technique performed by regional oximetry device to receive and display MAP values determined by an MPM device, as well as a corresponding example technique performed by the MPM device.

FIGS. 10A and 10B are a flow diagram and a conceptual block diagram illustrating an example technique performed by regional oximetry device to determine, display, and communicate MAP values to an MPM device using two algorithms.

FIGS. 11A and 11B are a flow diagram and a conceptual block diagram illustrating an example technique performed by regional oximetry device to determine, display, and communicate MAP values to an MPM device using one algorithm.

FIGS. 12A and 12B are a flow diagram and a conceptual block diagram illustrating an example technique performed by regional oximetry device to receive and display MAP values determined by an MPM device.

FIG. 13 is a flow diagram illustrating an example technique for synchronizing MAP values and oxygen saturation values.

FIG. 14 is a flow diagram illustrating an example regional oximetry device including processing circuitry configured to synchronize regional oxygen saturation (rSO₂) and MAP signals.

### DETAILED DESCRIPTION

This disclosure describes devices, systems, and techniques for presenting mean arterial pressure (MAP) values of a patient, e.g., for autoregulation monitoring. A system may include a regional oximetry device configured to present, via a display of the regional oximetry device, a set of MAP values of a patient for a time period. The system may also include a multi-parametric monitor (MPM) device different from the regional oximetry device and configured to present, via a display of the MPM device, a set of MAP values of the patient. In some cases, the regional oximetry device and the MPM device may present MAP values for the same time period (e.g., based on the same raw blood pressure signal and/or determined blood pressure values) that differ from each other. These differences may be due to, for example, the regional oximetry device and the MPM device using different algorithms to determine MAP values. Thus, even if the regional oximetry device and the MPM device determine MAP values for the same time period and using the same raw blood pressure signal, the regional oximetry device and the MPM device may display different MAP values.

If both the regional oximetry device and the MPM device are used to monitor a patient at the same time, e.g., during a medical procedure, then it may be undesirable for the regional oximetry device and the MPM device to present MAP values for the same time period that differ substantially. For example, a clinician may lose confidence in the MAP values presented by the devices or be uncertain of which set of MAP values to rely on during a medical procedure. The devices, systems, and techniques of this disclosure may help address these issues and improve the operation of a regional oximetry device and/or a MPM device by increasing the consistency between the MAP values presented by the regional oximetry device and MAP values presented by an MPM device.

The devices, systems, and techniques of this disclosure may enable more nuanced coordination between the presentation of MAP values by two devices. In contrast, a MPM device and regional oximetry device that merely use the same raw blood pressure signal to determine sets of MAP values, without any other coordination between the two devices, may result in larger differences between the MAP values presented by the MPM device and the regional oximetry device. The devices, systems, and techniques of this disclosure may reduce the likelihood that a clinician will receive different MAP values from the two devices. This may, for example, result in devices that provide the clinician with an improved user interface (including the MAP values), and reduce the time that the clinician takes to decide whether the blood pressure of the patient is in the intact region of autoregulation.

In some examples, the devices, systems, and techniques of this disclosure may also improve the accuracy of the regional oximetry device by synchronizing (in time) a set of MAP values and a set of regional oxygen saturation (rSO₂) values that the regional oximetry device uses to determine the autoregulation status of the patient. For example, the regional oximetry device may determine the set of MAP values based on a first physiological signal received from the patient and may determine the set of rSO₂ values based on a second physiological signal received from the patient, the first and second physiological signals being indicative of different physiological parameters of the patient. The two physiological signals may not necessarily be synchronized (in time) because of the different sensor circuitry for sensing the physiological parameters and generating the physiological signals change as a function of the respective physiological parameter. In addition, the regional oximetry device may use sampling windows of different time durations for each physiological signal, which may result in unsynchronized MAP and rSO₂ values. The MAP and rSO₂ values may be synchronized in time if the MAP and rSO₂ values are matched up exactly in time or represent values measured within less than one second of each other, such as within 0.5 seconds. In some examples, the MAP values may be offset from the rSO₂ values by a relative time delay, which may cause the regional oximetry device to determine an autoregulation status based on a MAP value at time X and a rSO₂ value at time X plus the relative time delay. This may result in a less accurate determination of a patient's autoregulation status. The devices, systems, and techniques of some examples described herein may help address these issues.

A regional oximetry device using synchronized MAP and rSO₂ values may provide a more accurate determination of autoregulation status, e.g., as compared to a device using unsynchronized MAP and rSO₂ values to determine autoregulation status. By synchronizing the MAP and rSO₂ values, the regional oximetry device may determine the autoregulation status of the patient based on more coherent data. For example, the regional oximetry device may determine cerebral oximetry index (COx) values for the MAP and rSO₂ values by determining a set of data points. Each data point may be associated with a MAP value at a particular time and a rSO₂ value at the particular time. If the MAP and rSO₂ values are synchronized, then each data point may be associated with a MAP value at a particular time and a rSO₂ value at the same particular time. In contrast, if the MAP and rSO₂ values are unsynchronized, then each data point may be associated with a MAP value at a particular time and a rSO₂ value at the particular time plus or minus a relative time delay. The relative time delay may cause the regional oximetry device to determine an autoregulation status of a patient using data representative of patient states at different times. Using data that represents patient states at different times may introduce error into a technique used to determine the autoregulation status. For example, using data representing patient states at different times can introduce error into the determination of COx values, which can be used to determine the autoregulation status of a patient.

The autoregulation status may be an indication that the cerebral autoregulation control mechanism of a patient is intact (e.g., functioning properly) or impaired. Cerebral blood flow (CBF) may be regulated over a range of systemic blood pressures by the CA control mechanism. This range may lie within the lower and upper limits of autoregulation (LLA and ULA), beyond which blood pressure drives CBF, and CA function may be considered impaired. One method to determine the limits of autoregulation (the LAs) noninvasively using near-infrared spectroscopy (NIRS) technology may be via the COx measure: a moving correlation index between MAP and rSO₂. In an intact region of CA, there may be no correlation between these variables whereas in an impaired region of CA, the correlation index should approximate unity. In practice, however, the data may be noisy and/or the intact region may exhibit a slightly positive relationship.

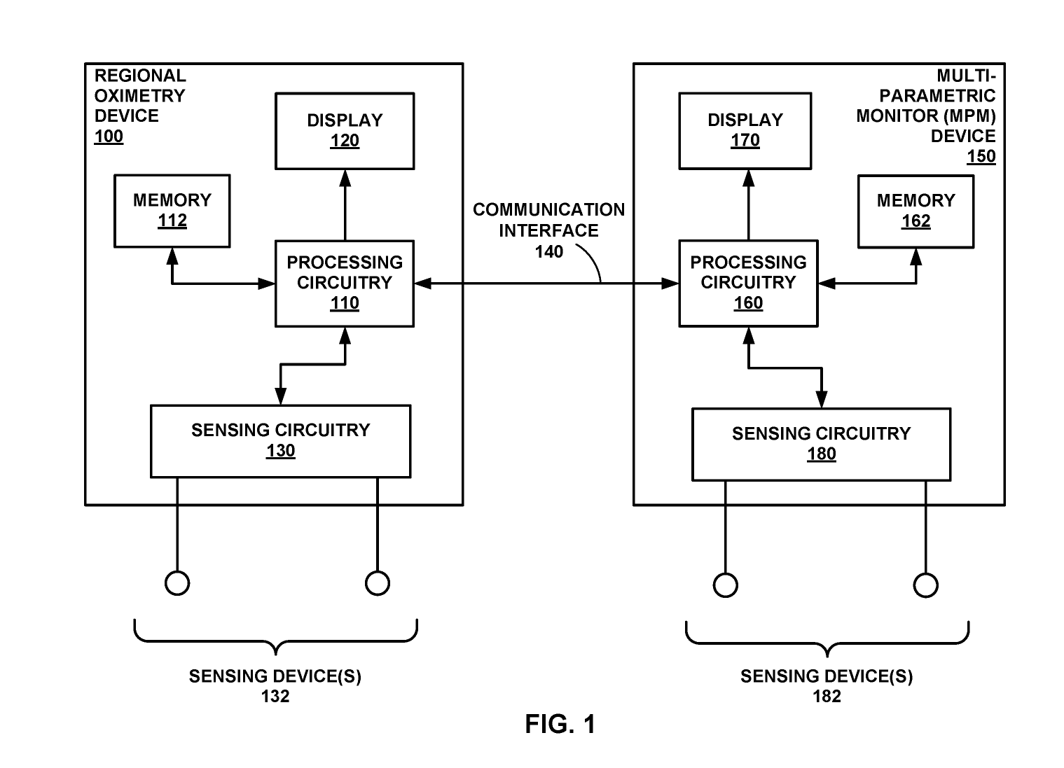

FIG. 1 is a conceptual block diagram illustrating an example patient monitoring system that includes an example regional oximetry device 100 and an example multi-parametric (MPM) device 150. Regional oximetry device 100 includes processing circuitry 110, memory 112, display 120, sensing circuitry 130, and sensing device(s) 132. MPM device 150 includes processing circuitry 160, display 170, sensing circuitry 180, and sensing device(s) 182. In some examples, regional oximetry device 100 may be configured to determine and display the autoregulation status of a patient, e.g., during a medical procedure or for more long-term monitoring, such as monitoring of prenatal infants. A clinician may receive information regarding the autoregulation status of a patient via display 120 and adjust treatment or therapy to the patient based on the autoregulation status information.

Each of processing circuitry 110 and 160 may include one or more processors. Processing circuitry 110 and 160 may include any combination of integrated circuitry, discrete logic circuitry, analog circuitry, such as one or more microprocessors, digital signal processors (DSPs), application specific integrated circuits (ASICs), or field-programmable gate arrays (FPGAs). In some examples, processing circuitry 110 and 160 may include multiple components, such as any combination of one or more microprocessors, one or more DSPs, one or more ASICs, or one or more FPGAs, as well as other discrete or integrated logic circuitry, and/or analog circuitry.

Displays 120 and 170 may be configured to present information to a user (e.g., a clinician). One or both of displays 120 and 170 may be configured to present a graphical user interface to a user, where each graphical user interface may include indications of one or more physiological parameters. For example, processing circuitry 110 may be configured to present MAP values and an indication of autoregulation status of a patient via display 120. In some examples, if processing circuitry 110 determines that the autoregulation status of the patient is impaired, then processing circuitry 110 may present a notification (e.g., an alert) indicating the impaired autoregulation status via display 120. As another example, processing circuitry 110 or processing circuitry 160 may present, via the respective displays 120 and 170, estimates of rSO₂ for a patient, an estimate of the blood oxygen saturation (SpO₂) determined by the respective processing circuitry 110 or 160, pulse rate information, respiration rate information, blood pressure, any other patient parameters, or any combination thereof.

Displays 120 and 170 may each include a monitor, cathode ray tube display, a flat panel display such as a liquid crystal (LCD) display, a plasma display, or a light emitting diode (LED) display, personal digital assistant, mobile phone, tablet computer, laptop computer, any other suitable display device, or any combination thereof. Devices 100 and 150 may also include means for projecting audio to a user, such as speaker(s).

Sensing circuitry 130 and 180 may be configured to receive physiological signals sensed by respective sensing device(s) 132 and 182 and communicate the physiological signals to the respective processing circuitry 110 and 160. Sensing device(s) 132 and 182 may include any sensing hardware configured to sense a physiological parameter of a patient, such as, but not limited to, one or more electrodes, optical receivers, blood pressure cuffs, or the like. Sensing circuitry 130 and 180 may convert the physiological signals to usable signals for processing circuitry 110 and 160. Sensing circuitry 130 and 180 may receive signals indicating physiological parameters from a patient, such as, but not limited to, blood pressure, rSO₂, heart rate, and respiration. Sensing circuitry 130 and 180 may include, but are not limited to, blood pressure sensing circuitry, rSO₂ sensing circuitry, heart rate sensing circuitry, temperature sensing circuitry, electrocardiography (ECG) sensing circuitry, electroencephalogram (EEG) sensing circuitry, or any combination thereof. In some examples, sensing circuitry 130 and 180 and/or processing circuitry 110 and 160 may include signal processing circuitry such as an analog-to-digital converter.

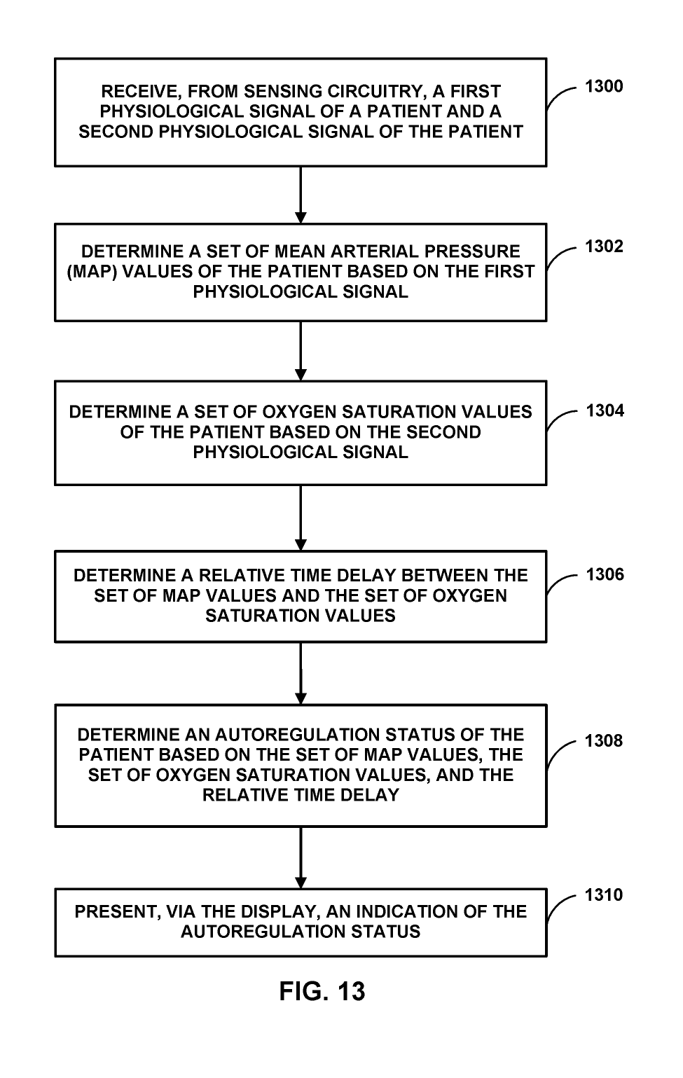

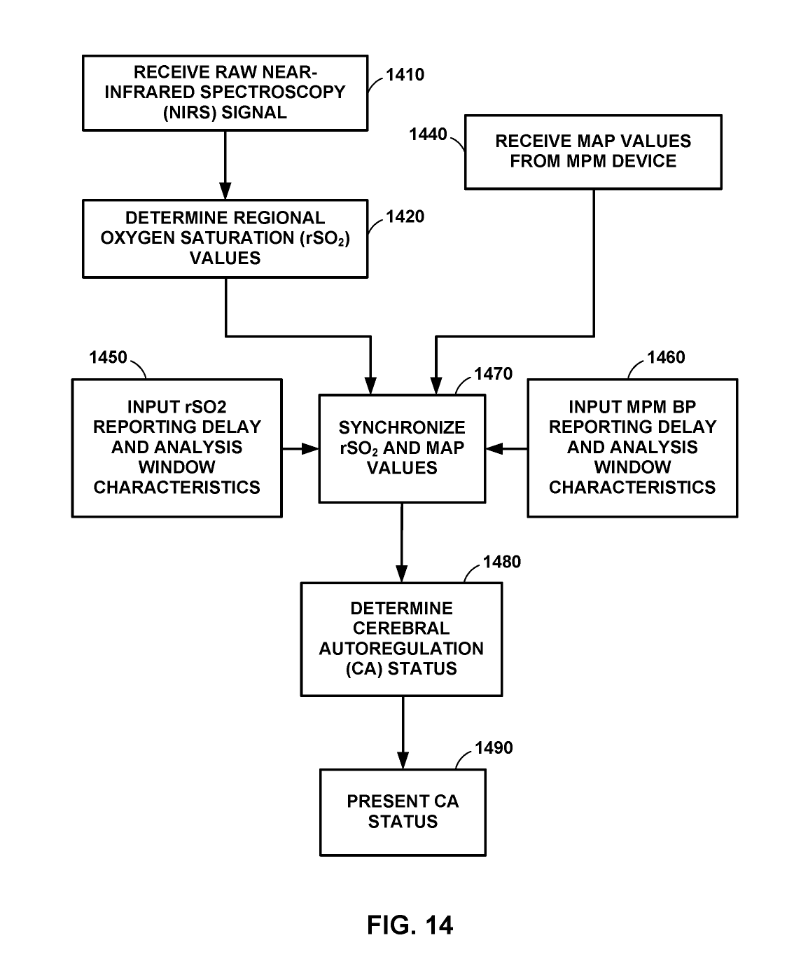

Processing circuitry 110 may be configured to receive one or more physiological signals generated by sensing circuitry 130. The physiological signals may include a signal indicating blood pressure and/or a signal indicating oxygen saturation of a patient. Processing circuitry 110 may be configured to determine a first set of MAP values of a patient for a time period based on a first physiological signal. Processing circuitry 110 may also be configured to determine a set of rSO₂ values based on a second physiological signal indicative of a physiological parameter different from the first physiological parameter. For example, the first physiological parameter may be indicative of blood pressure of a patient and the second physiological parameter may be indicative of regional oxygen saturation of a target tissue site of the patient. In some examples, processing circuitry 110 is configured to determine a relative time delay between the MAP values and the oxygen saturation values. As discussed in further detail below with respect to FIGS. 13 and 14, processing circuitry 110 may use the relative time delay to synchronize the MAP values and the oxygen saturation values and/or to determine the autoregulation status of the patient.

Processing circuitry 110 may be configured to determine an autoregulation status of the patient based on the first set of MAP values using any suitable technique. For example, processing circuitry 110 may be configured to determine a set of COx values based on the MAP and rSO₂ values, and determine the LLA and ULA for the patient based on the set of COx values. Processing circuitry 110 may be configured to then determine the autoregulation status of the patient based on whether a current MAP value is between or outside of the LLA and the ULA. Additional example details of determining LA's and autoregulation status may be found in commonly assigned U.S. Patent Application Publication No. 2018/0014791 filed on Jul. 13, 2017, and entitled “Systems and Methods of Monitoring Autoregulation,” and commonly assigned U.S. Provisional Patent Application No. 62/510,303 filed on May 24, 2017, and entitled “Determining a Limit of Autoregulation,” the entire contents of each of which are incorporated herein by reference.

In some examples, processing circuitry 110 is configured to display determined MAP values of a patient, for example, in real-time or nearly in real-time (e.g., delays of less than five or ten seconds), via display 120. Processing circuitry 160 of MPM device 150 may also be configured to display determined MAP values of the patient via display 170. The MAP values used by processing circuitry 110 to determine a regional oximetry value may differ from the MAP values presented on display 170, though the MAP values presented on display 170 may be representative of MAP values for the same, overlapping time periods (e.g., a particular point in time, such as 12:00 p.m. on the same day) as those used by processing circuitry 110 to determine a regional oximetry value. The MAP values may be different because processing circuitry 110 may be configured to use a different algorithm to determine MAP values than an algorithm used by processing circuitry 160 to determine MAP values for presentation via display 170.

Processing circuitry 110 may be configured to use an algorithm to determine MAP values that is specifically tailored to autoregulation monitoring because the blood pressure signals of patients that undergo autoregulation monitoring may exhibit unique waveforms. In some examples, to determine MAP values, processing circuitry 110 may use an algorithm that is more robust to blood pressure artifacts than an algorithm used by processing circuitry 160 to determine MAP values. Processing circuitry 160 may be configured to report MAP values even when such artifacts are present. Examples of blood pressure artifacts include changes in blood pressure due to electrocautery, damping of blood pressures due to catherization, changes in sensed blood pressure due to probe movement, and changes in sensed blood pressure due to line flushing. Processing circuitry 160 may be more likely than processing circuitry 110 to incorrectly determine MAP values during artifact events, and incorrect MAP values may adversely affect autoregulation determinations. When an artifact event occurs such as a line flushing event, a clinician may know about the artifact event and ignore the presentation of MAP values on a MPM display 170. Presenting incorrect MAP values and/or incorrect autoregulation status information can reduce clinician confidence in a monitoring device. Other sources of differences in MAP values determined by processing circuitry 110 and processing circuitry 160 include one or more of window averaging, rounding to nearest integer, and incorrect implementation of the integration of the pulses, which can lead to an error that depends on the heart-rate of the subject.

In some examples, processing circuitry 110 of regional oximetry device 100 is configured to present, via display 120, a set of MAP values that is coherent with the MAP values presented on display 170 by processing circuitry 160 of MPM device 150. For example, the set of MAP values that is presented by processing circuitry 110 may be coherent and consistent because processing circuitry 110 or 160 may determine and communicate a set of MAP values to the other of processing circuitry 110 and 160 via communication interface 140. In this way, both of regional oximetry device 100 and MPM device 150 may present the same set of MAP values via the respective displays 120, 170.

In other examples, each of processing circuitry 110 and 160 may determine and present separate sets of MAP values based on the same raw blood pressure signal generated by sensing circuitry 180 of MPM device 150 or based on different raw blood pressure signals, e.g., generated by respective sensing circuitries 130 and 180 or by another sensing device separate from regional oximetry device 100 and MPM device 150. In some of these examples, processing circuitry 110 may determine a set of MAP values to mimic the determination of MAP by processing circuitry 160 by using an algorithm that is similar to, or the same as, the algorithm used by processing circuitry 160. Thus, processing circuitry 110 and 160 may independently determine sets of MAP values (for the same time period) using the same or similar algorithms. By mimicking the determination of MAP by processing circuitry 160, processing circuitry 110 may improve the consistency between the MAP values presented on display 120 and the MAP values presented on display 170 and, therefore, generate an improved graphical user interface that includes MAP values for a patient.

Using any of the techniques or combination of techniques described above, devices 100 and 150 may share MAP values with each other and/or mimic the presentation of MAP values (mimic the values presented by the other device) to reduce or eliminate the differences between the MAP values presented by the different devices 100, 150. By presenting coherent MAP values on displays 120 and 170, processing circuitry 110 and 160 may minimize or eliminate any inconsistencies between the two sets of MAP values, resulting in an improved graphical user interface. The presentation of coherent MAP values on displays 120 and 170 may result in increased confidence in the displayed MAP values by a clinician viewing the presented sets of MAP values, which may lead to more informed decision making by the clinician.

In some examples, the MAP values displayed by processing circuitry 110 of regional oximetry device 100 via display 120 are a second set of MAP values of the patient that represents MAP values determined for the same time period as the first set of MAP values, the first set being the MAP values determined by processing circuitry 110 and used to determine an autoregulation status of a patient. The processing circuitry 110 may determine the second set of MAP values to display using one or more techniques described herein. In some examples, processing circuitry 110 may be configured to receive the second set of MAP values from MPM device 150 or processing circuitry 110 may be configured to determine the second set of MAP values based on the first physiological signal received from sensing circuitry 130. If processing circuitry 110 determines the second set of MAP values based on the first physiological signal, then processing circuitry 110 may be configured to use the same algorithm or a different algorithm for determining both the first set and the second set of MAP values. Accordingly, processing circuitry 110 may be configured to use the same set of MAP values for determining an autoregulation status and for presenting via display 120.

Processing circuitry 110 may be configured to determine the second set of MAP values to mimic determination of MAP values by MPM device 150, where the second set of MAP values may be different than the first set of MAP values for determining autoregulation status, even if the first and second sets of MAP values represent MAP values for the same, overlapping (e.g., entirely overlapping) time periods. In some examples, processing circuitry 110 may receive the second set of MAP values from MPM device 150. Processing circuitry 110 may be configured to present the received second set of MAP values via display 120 and, in some examples, may determine the autoregulation status based on the received second set of MAP values. In other examples, however, processing circuitry 110 may present the received second set of MAP values via display 120 and determine the autoregulation status based on the first set of MAP values.

MPM device 150 may be configured to determine physiological parameters of a patient and present the determined physiological parameters via display 170. For example, processing circuitry 160 may be configured to present determined MAP values and/or oxygen saturation values via display 170. MPM device 150 may also be configured to present determined values of other physiological parameters such as the patient's heart rate and/or hemoglobin concentration. As described herein, in some examples, MPM device 150 may be configured to present MAP values determined by processing circuitry 110 of regional oximetry device 100, while in other examples, MPM device 150 may be configured to present MAP values determined by processing circuitry 160.

Processing circuitry 160 may be configured to determine physiological parameter values of a patient to present on display 170, where the values may be for the same or different types of physiological parameters monitored and displayed by regional oximetry device 100. For example, processing circuitry 160 may be configured to present values for physiological parameters such as regional oxygen saturation, pulse rate, respiration rate, respiration effort, body temperature, blood pressure, blood oxygen saturation (e.g., arterial, venous, or both), hemoglobin concentration (e.g., oxygenated, deoxygenated, and/or total), any other suitable physiological parameters, or any combination thereof, whereas processing circuitry 110 may be configured to present values for physiological parameters such as regional oxygen saturation and MAP.

For a given type of physiological parameter, such as MAP, the physiological parameter values presented on display 170 of MPM device 150 may be different than the physiological parameter values presented on display 120 of regional oximetry device 100. For example, as discussed above, processing circuitry 110 and 160 may be configured to use different algorithms for determining a physiological parameter such as MAP, such that processing circuitry 110 and 160 may present different MAP values via displays 120 and 170 for the same time period. Even if sensing circuitry 130, 180, via respective sensing devices 132 and 182, receive the same physiological signal indicative of MAP, processing circuitry 110 may be configured to determine a first set of MAP values using a first algorithm, and processing circuitry 160 may be configured to determine a second set of MAP values using a second algorithm different from the first algorithm. The techniques described above may help synchronize the MAP values displayed by displays 120, 170 of devices 100, 150, respectively.

In addition to a physiological signal indicating blood pressure, in some examples, processing circuitry 110 may also receive one or more physiological signals indicating other physiological parameters such as oxygen saturation. Processing circuitry 110 may be configured to determine other physiological parameter values based on the additional physiological signals. For example, processing circuitry 110 may be configured to use the determined MAP values and the determined physiological parameter values in order to determine an autoregulation status of the patient. In some examples, processing circuitry 110 determines the autoregulation status of a patient based on a set of MAP values for a time period and a set of oxygen saturation values for the same time period.

A patient state, as indicated by sensed physiological signals, may change relatively rapidly over time. Thus, if processing circuitry 110 determines an autoregulation status of a patient based on physiological signals that do not coincide in time, then the determined autoregulation status may not be accurate. In some examples, processing circuitry 110 may be configured to synchronize (in time) a blood-pressure physiological signal and an oxygen-saturation physiological signal, such that an autoregulation status of a patient is determined based on physiological signals indicative of the same patient state. In some examples, processing circuitry 110 is also configured to synchronize other sets of two or more physiological signals in addition to or instead of blood pressure and oxygen-saturation signals, where each set may include signals such as, but not limited to, blood pressure signals, oxygen saturation signals, blood volume signals, hemoglobin-related signals, and/or other physiological signals. Processing circuitry 110 may be configured to determine a relative time delay between the MAP values and the oxygen saturation values and use the relative time delay to determine the autoregulation status. Processing circuitry 110 may be configured to shift the set of MAP values and/or the set of rSO₂ values before determining the autoregulation status of the patient.

The relative time delay may be caused by the hardware and/or software of regional oximetry device 100. For example, regional oximetry device 100 may use different circuitry for receiving the first physiological signal and the second physiological signal, which may result in different delays in the communication of the physiological signals from the patient to processing circuitry 110. In addition to or instead of the time delay caused by the circuitry, the relative time delay may result from processing the physiological signals, the MAP values, and the rSO₂ values. In some examples, processing circuitry 110 may be configured to use sampling windows with different time durations for each physiological signal, and the different time durations may cause a relative time delay between the MAP values and the oxygen saturation values.

Memory 112 and memory 162 may be configured to store measurements of physiological parameters, MAP values, rSO₂ values, COx values, and value(s) of an LLA and/or a ULA, for example. Memory 112 and 162 may also be configured to store data such as threshold levels for physiological parameters and threshold levels for signal quality metrics. In some examples, memory 112 and 162 may store program instructions, which may include one or more program modules, which are executable by processing circuitry 110 and 160. When executed by processing circuitry 110 and 160, such program instructions may cause processing circuitry 110 and 160 to provide the functionality ascribed to it herein. The program instructions may be embodied in software, firmware, and/or RAMware. Memory 112 and 162 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media.

Communication interface 140 may enable device 100 to exchange information with device 150 and/or other external devices. Communication interface 140 may include any suitable hardware, software, or both, which may allow device 100 to communicate external devices such as electronic circuitry, a device, a network, a server or other workstations, a display, or any combination thereof. Communication interface 140 may include one or more receivers, transmitters, transceivers, antennas, plug-in connectors, ports, communications buses, communications protocols, device identification protocols, any other suitable hardware or software, or any combination thereof. Communication interface 140 may be configured to enable wired communication (e.g., using USB, RS-232, Ethernet, coaxial, or other standards), wireless communication (e.g., using Wi-fi, infrared, WiMax, Bluetooth, universal serial bus (USB), or other standards), or both. For example, communication interface 140 may be configured using a USB protocol (e.g., USB 2.0, USB 3.0), and may be configured to couple to other devices (e.g., remote memory devices storing templates) using a four-pin USB standard Type-A connector (e.g., plug and/or socket) and cable. In some examples, communication interface 140 may include an internal bus such as, for example, one or more slots for insertion of expansion cards.

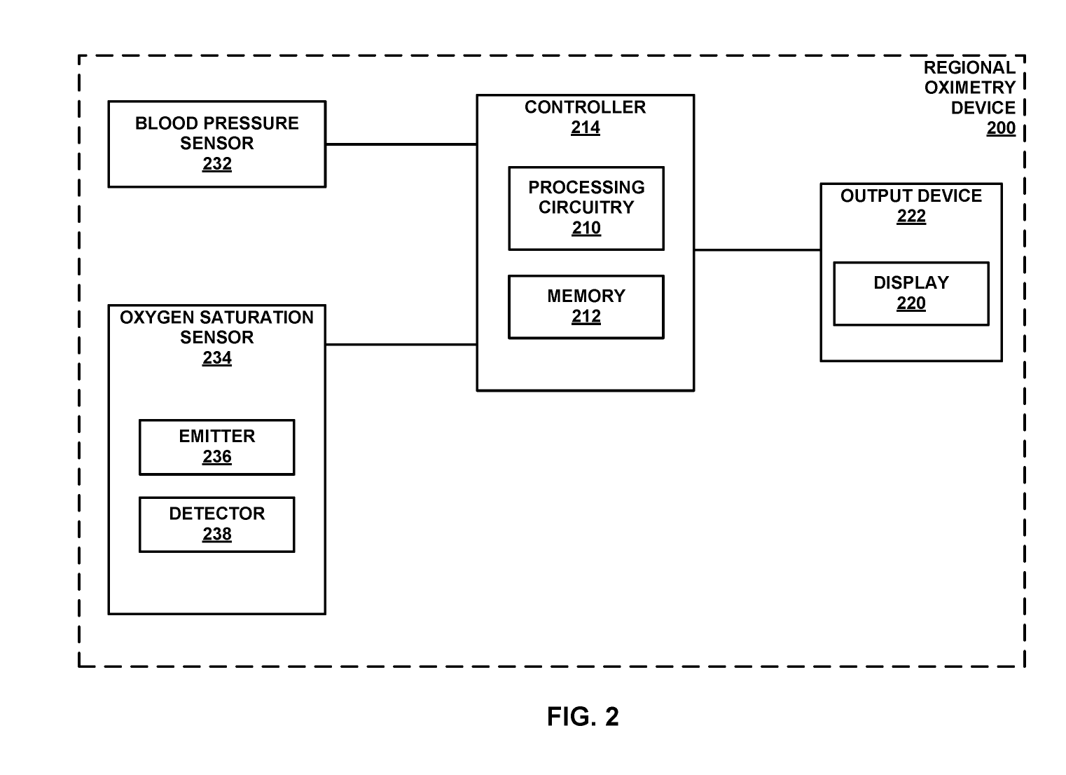

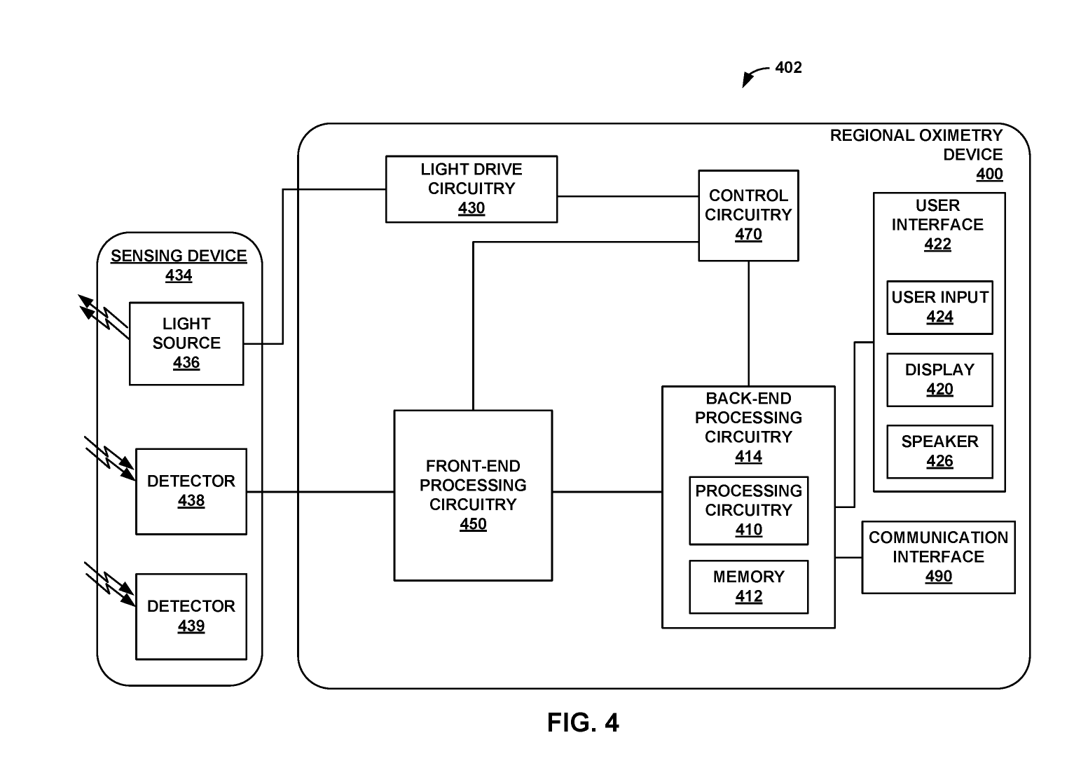

FIG. 2 is a conceptual block diagram illustrating an example regional oximetry device 200 configured to monitor the autoregulation status of a patient. As shown, regional oximetry device 200 includes blood pressure sensor 232, oxygen saturation sensor 234 (e.g., a regional oxygen saturation sensor), controller 214, and output device 222. Regional oximetry device 200 is an example of regional oximetry device 100 shown in FIG. 1, and blood pressure sensor 232 and oxygen saturation sensor 234 are examples of sensing devices 132 shown in FIG. 1. In some examples, regional oximetry device 200 may or may not include blood pressure sensor 232. Regional oximetry device 400, depicted in FIG. 4, is an example of a regional oximetry device without a blood pressure sensor.

Blood pressure sensor 232 may be any sensor or device configured to obtain the patient's blood pressure (e.g., arterial blood pressure). For example, blood pressure sensor 232 may include a blood pressure cuff for non-invasively monitoring blood pressure or an arterial line for invasively monitoring blood pressure. In certain examples, blood pressure sensor 232 may include one or more pulse oximetry sensors. In some such cases, the patient's blood pressure may be derived by processing time delays between two or more characteristic points within a single plethysmography (PPG) signal obtained from a single pulse oximetry sensor.

Additional example details of deriving blood pressure based on a comparison of time delays between certain components of a single PPG signal obtained from a single pulse oximetry sensor are described in commonly owned U.S. Patent Application Publication No. 2009/0326386 filed Sep. 30, 2008, and entitled “Systems and Methods for Non-Invasive Blood Pressure Monitoring,” the entire content of which is incorporated herein by reference. In other cases, the patient's blood pressure may be continuously, non-invasively monitored via multiple pulse oximetry sensors placed at multiple locations on the patient's body. As described in U.S. Pat. No. 6,599,251, entitled “Continuous Non-invasive Blood Pressure Monitoring Method and Apparatus,” the entire content of which is incorporated herein by reference, multiple PPG signals may be obtained from the multiple pulse oximetry sensors, and the PPG signals may be compared against one another to estimate the patient's blood pressure. Regardless of its form, blood pressure sensor 232 may be configured to generate a blood pressure signal indicative of the patient's blood pressure (e.g., arterial blood pressure) over time. As discussed in more detail below, blood pressure sensor 232 may provide the blood pressure signal to controller 214 or to any other suitable processing device to enable evaluation of the patient's autoregulation status.

In some examples, oxygen saturation sensor 234 is a regional oxygen saturation sensor configured to generate an oxygen saturation signal indicative of blood oxygen saturation within the venous, arterial, and/or capillary systems within a region of the patient. For example, oxygen saturation sensor 234 may be configured to be placed on the patient's forehead and may be used to determine the oxygen saturation of the patient's blood within the venous, arterial, and/or capillary systems of a region underlying the patient's forehead (e.g., in the cerebral cortex).

In such cases, oxygen saturation sensor 234 may include emitter 236 and multiple detectors 238. Emitter 236 may include at least two light emitting diodes (LEDs), each configured to emit at different wavelengths of light, e.g., red or near infrared light. In some examples, light drive circuitry (e.g., within sensor 234 or controller 214) may provide a light drive signal to drive emitter 236 and to cause emitter 236 to emit light. In some examples, the LEDs of emitter 236 emit light in the range of about 600 nanometers (nm) to about 1000 nm. In a particular example, one LED of emitter 236 is configured to emit light at about 730 nm and the other LED of emitter 236 is configured to emit light at about 810 nm. Other wavelengths of light may also be used in other examples.

One of detectors 238 is positioned relatively “close” (e.g., proximal) to emitter 236 and one of detectors 238 is positioned relatively “far” (e.g., distal) from emitter 236. Light intensity of multiple wavelengths may be received at both the “close” and the “far” detectors 238. For example, if two wavelengths are used, the two wavelengths may be contrasted at each location and the resulting signals may be contrasted to arrive at a regional saturation value that pertains to additional tissue through which the light received at the “far” detector passed (tissue in addition to the tissue through which the light received by the “close” detector passed, e.g., the brain tissue), when it was transmitted through a region of a patient (e.g., a patient's cranium). Surface data from the skin and skull may be subtracted out, to generate a regional oxygen saturation (rSO₂) signal for the target tissues over time. Oxygen saturation sensor 234 may provide the regional oxygen saturation signal to controller 214 or to any other suitable processing device to enable evaluation of the patient's autoregulation status.

In operation, blood pressure sensor 232 and oxygen saturation sensor 234 may each be placed on the same or different parts of the patient's body. For example, blood pressure sensor 232 and oxygen saturation sensor 234 may physically separate from each other and separately placed on the patient. As another example, blood pressure sensor 232 and oxygen saturation sensor 234 may in some cases be part of the same sensor or supported by a single sensor housing. For example, blood pressure sensor 232 and oxygen saturation sensor 234 may be part of an integrated oximetry system configured to non-invasively measure blood pressure (e.g., based on time delays in a PPG signal) and regional oxygen saturation. One or both of blood pressure sensor 232 or oxygen saturation sensor 234 may be further configured to measure other parameters, such as hemoglobin, respiratory rate, respiratory effort, heart rate, saturation pattern detection, response to stimulus such as bispectral index (BIS) or electromyography (EMG) response to electrical stimulus, or the like. While an example regional oximetry device 200 is shown in FIG. 2, the components illustrated in FIG. 2 are not intended to be limiting. Additional or alternative components and/or implementations may be used in other examples.

In some examples, signals generated by sensors 232 and 234 may be offset in time. For example, each of sensors 232 and 234 may have a different delay in receiving, processing, and/or delivering the respective physiological signals to controller 214. The difference between the delay in controller 214 receiving a first physiological signal from blood pressure sensor 232 and the delay in controller 214 receiving a second physiological signal from oxygen saturation sensor 232 may result in a relative time delay between the determined MAP values and oxygen saturation values of a patient. As one example, at a particular time, sensor 232 may deliver a signal to controller 214 that indicates blood pressure one hundred milliseconds ago. At the same particular time, sensor 234 may deliver a signal to controller 214 that indicates oxygen saturation one second ago. If controller 214 determines an index or measure of cerebral autoregulation, such as a correlation index (e.g., COx index) or another measure, based on the two unsynchronized data sets, then the relative time delay may cause the determination of the COx index by controller 214 to be inaccurate or at least less accurate compared to instances in which there is no relative time delay. The COx index may be inaccurate or less accurate because the two signals may represent patient states at different times, particularly if the patient state is changing relatively quickly over time.

Even if sensors 232 and 234 deliver signals to controller 214 with no relative time delay, inaccuracies may arise if controller 214 uses algorithms for determining mean arterial pressure and oxygen saturation with different-sized sampling windows. For example, controller 214 may be configured to determine mean arterial pressure based on a sampling window of ten seconds and determine oxygen saturation based on a sampling window of five seconds. The different-sized sampling windows may cause the MAP values and the oxygen saturation values to not be matched up exactly in time, which may cause controller 214 to determine COx values and an autoregulation status based on unsynchronized data.

In some examples, regional oximetry device is configured to synchronize (in time) signals received from blood pressure sensor 232 and oxygen saturation sensor 234. To synchronize the signals received and processed by controller 214, regional oximetry device 200 may be configured to receive user input indicating a model number of blood pressure sensor 232 or another blood pressure measurement device that is connected to regional oximetry device 200. In some examples, there may be a separate blood pressure measurement device that receives a signal from blood pressure sensor 232, determines the MAP values, and delivers the MAP values to controller 214. For example, regional oximetry device 200 may include an input device such as a keyboard or a touchscreen that allows the user to input the model number of the blood pressure equipment, and controller 214 may determine the time delay associated with the blood pressure equipment. A particular model of blood pressure equipment may be associated with a known time delay in a memory (e.g., memory 212) of device 200 or another device communicatively coupled to device 200. Controller 214 may use the determined time delay to synchronize the MAP values with the oxygen saturation value in order to improve the accuracy of the COx index. Controller 214 may determine the COx index, and ultimately the autoregulation status, based on the MAP values, the oxygen saturation values, and the relative time delay between the MAP values and the oxygen saturation values.

In some examples, controller 214 may determine the relative time delay based on the sampling windows for mean arterial pressure and oxygen saturation. If the durations of the sampling windows are ten and five seconds, respectively, for mean arterial pressure and oxygen saturation, then the centers of the sampling windows may be offset by two-and-one-half seconds. Using these example durations, controller 214 may determine a MAP value based on a waveform of the blood pressure signal over the previous ten seconds and determine an rSO₂ value based on a waveform of the oxygen saturation signal over the previous five seconds. Controller 214 may determine a relative time delay of two-and-one-half seconds in order to align the centers of the sampling windows. When controller 214 determines an aggregated value from a sampling window, the center of a sampling window is effectively the source of the aggregated value if the sampling and weighting is constant across the sampling window.

In some examples, controller 214 may be configured to determine the relative time delay between the data sets in real-time. Controller 214 may determine the relative time delay by determining a cross-correlation function for the MAP values and the oxygen saturation values.

Controller 214 may then determine the time lag at which the cross-correlation function has a maximum amplitude, which may indicate the relative time delay between the MAP and oxygen saturation values. Controller 214 may use the time lag to synchronize the set of MAP values and the set of oxygen saturation values. In some examples, controller 214 may synchronize the two sets by adding or subtracting the determined delay from the time (e.g., timestamp) associated with each value in the set. For example, controller 214 may store a multi-dimensional data structure such as an array to memory 212, where the data structure associates each MAP value with a time of measurement. Controller 214 may adjust (e.g., shift) the associated time by the determined delay in order to synchronize the set of MAP values with the set of oxygen saturation values. When synchronized, the MAP values for a particular time period better correspond in time to the oxygen saturation values observed for the patient for the same time period. Controller 214 may then use the synchronized data sets to determine COx values and the limit(s) of autoregulation.

As noted above, blood pressure sensor 232 may be configured to provide the blood pressure signal to controller 214, and oxygen saturation sensor 234 may be configured to provide the oxygen saturation signal to controller 214. In certain examples, controller 214 is an electronic controller having electrical circuitry configured to process the various received signals. For example, controller 214 may be configured to process the blood pressure signal and the oxygen saturation signal to evaluate the patient's cerebral autoregulation status. Although blood pressure sensor 232 and oxygen saturation sensor 234 may be configured to provide their respective signals or data directly to controller 214, in certain examples, the signals or data obtained by blood pressure sensor 232 and/or oxygen saturation sensor 234 may be provided to one or more intermediate processing devices (e.g., specialized monitor, such as a blood pressure monitor or an oxygen saturation monitor, or the like), which may in turn provide processed signals or data to controller 214.

Controller 214 may be configured to determine COx values based on the blood pressure signal and the oxygen saturation signal. The COx index may be indicative of vascular reactivity, which is related to cerebral blood vessels' ability to control proper blood flow, via vasoconstriction (a narrowing of the blood vessel) and/or vasodilation (expansion of the blood vessel), for example. Thus, the COx index is also indicative of whether the patient's autoregulation is impaired. Controller 214 may derive the COx index by determining a linear correlation between blood pressure measurements and oxygen saturation measurements. The linear correlation may be based on a Pearson coefficient, for example. The Pearson coefficient may be defined as the covariance of the measured blood pressure (e.g., arterial blood pressure) and oxygen saturation divided by the product of their standard deviations. The result of the linear correlation may be a regression line between oxygen saturation measurements and blood pressure measurements, and the slope of the regression line may be indicative of the patient's autoregulation status. In one example implementation, a regression line with a relatively flat or negative slope (e.g., regional oxygen saturation increases after blood pressure decreases) may suggest that cerebral autoregulation of the patient is working properly, while a regression line with a positive slope (e.g., regional oxygen saturation remains the same or decreases after blood pressure decreases) may suggest that the cerebral autoregulation of the patient is impaired. Controller 214 may be configured to generate a notification in response to determining that the autoregulation status of a patient is impaired. Controller 214 may then be configured to present the notification via display 220.

Controller 214 may determine a value of the COx index, which may be between −1 and 1, inclusive, where −1 represents total negative correlation, +1 represents total positive correlation, and 0 represents the absence of correlation between the blood pressure measurements and the oxygen saturation measurements. Thus, COx values between −1 and 0 may suggest that cerebral autoregulation of a patient is working properly, while COx values between 0 and 1 may suggest that the cerebral autoregulation of the patient is impaired. In some cases, a predetermined threshold between 0 and 1 may be utilized to determine whether the patient's autoregulation is impaired. For example, in some examples, controller 214 may be configured to determine that the patient's autoregulation is impaired when the COx value is greater than 0.1, 0.2, 0.3, 0.4, 0.5, 0.6, 0.7, 0.8, or 0.9. Accordingly, controller 214 may be configured to determine the COx value and/or the patient's autoregulation status based on the linear correlation between the blood pressure measurements and oxygen saturation measurements obtained by the blood pressure sensor 232 and the oxygen saturation sensor 234, respectively.

In the illustrated example, controller 214 includes processing circuitry 210 and memory 212, which are examples of processing circuitry 110 and memory 112 shown in FIG. 1. Controller 214 may also include one or more storage devices, such as memory 212. Processing circuitry 210 may be used to execute software, such as software for carrying out any of the techniques disclosed herein, such as processing the blood pressure signals and/or oxygen saturation signals, determining relative time delays, determining cross-correlation functions, determining COx values, synchronizing signals and data values, and carrying out appropriate remedial actions, and so forth.

Memory 212 may store a variety of information and may be used for various purposes. For example, memory 212 may store processor-executable instructions (e.g., firmware or software) for processing circuitry 210 to execute, such as instructions for carrying out any of the techniques discloses herein, such as processing blood pressure signals and/or oxygen saturation signals, determining relative time delays, determining cross-correlation functions, determining COx values, synchronizing signals and data values, and/or taking appropriate remedial actions. Memory 212 may store data (e.g., the blood pressure signal, the oxygen saturation signal, the COx, the relative time delay, the time delays for sensors 232 and 234 and other equipment, and the like), instructions (e.g., software or firmware for processing the blood pressure signal and/or the oxygen saturation signal, determining the COx and/or the relative time delay, and/or taking appropriate remedial actions), predetermined thresholds, and any other suitable data.

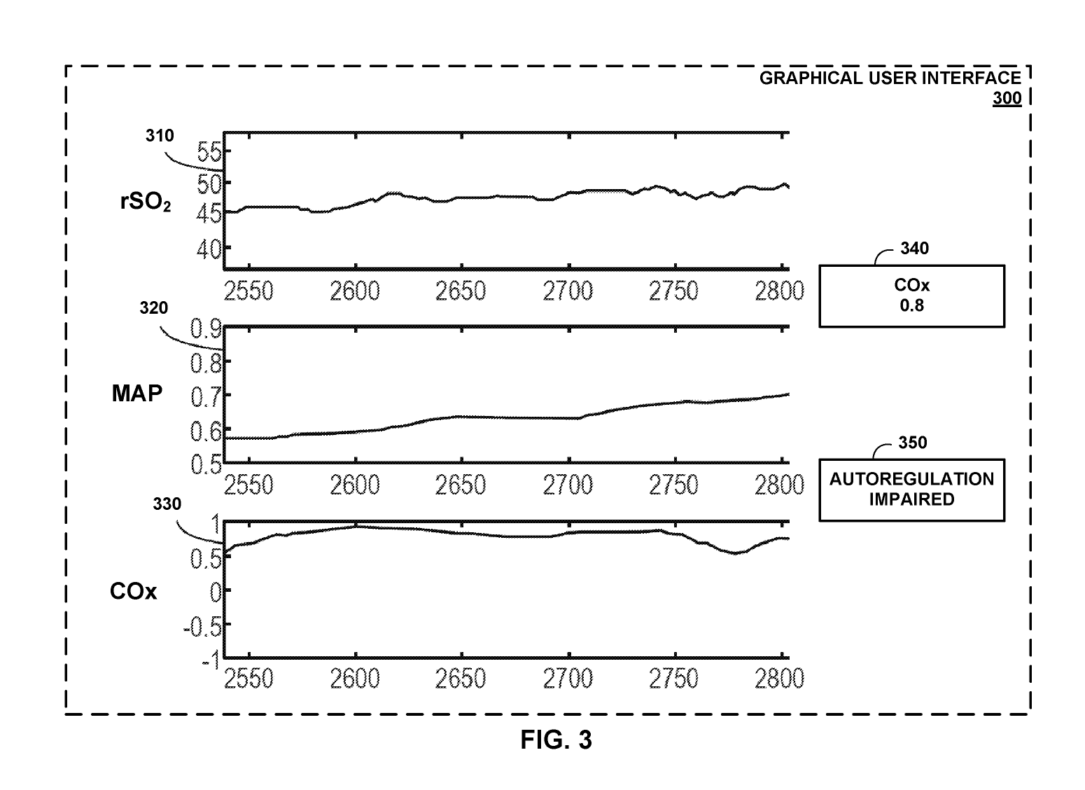

Regional oximetry device 200 includes output device 222, which includes display 220, which is an example of display 120 shown in FIG. 1. In some examples, controller 214 may be configured to provide signals indicative of the patient's autoregulation status to output device 222. As discussed in more detail below, controller 214 may be configured to present, via output device 222, a visual, audible, or somatosensory notification (e.g., an alarm signal) indicative of the patient's autoregulation status. Output device 222 may include any device configured to receive signals (e.g., the signal indicative of the patient's autoregulation status, the alarm signal, or the like) from controller 214 and visually, audibly, and/or somatosensorily output information indicative of the patient's autoregulation status (e.g., the COx value, the COx signal, an alarm, or the like). For instance, output device 222 can include display 220 configured to provide a visual representation of the patient's autoregulation status and/or the alarm signal as determined by controller 214 (see, e.g., graphical user interface 300 shown in FIG. 3). Additionally or alternatively, output device 222 may include an audio device configured to provide sounds in accordance with the alarm signal, the patient's autoregulation status, or both. Output device 222 may be any suitable device for conveying such information, including a computer workstation, a server, a desktop, a notebook, a laptop, a handheld computer, a mobile device, or the like. In some examples, controller 214 and output device 222 may be part of the same device or supported within one housing (e.g., a computer or monitor).

FIG. 3 illustrates an example graphical user interface 300 including autoregulation information. Processing circuitry 110 of regional oximetry device 100 (FIG. 1), processing circuitry 160 of MPM device 150 (FIG. 1), or processing circuitry of another device may be configured to generate graphical user interface 300 and present it via a respective display, e.g. display 120 or display 170. Thus, although processing circuitry 110 and display 120 of regional oximetry device 100 are primarily referred to throughout the description of FIG. 3, in other examples, graphical user interface 300 may be generated by and/or displayed by another device, such as MPM device 150.

FIG. 3 is an example of a presentation by processing circuitry 110 on display 120 shown in FIG. 1. Graphical user interface 300 may be configured to display various information related to the COx index. As shown, graphical user interface 300 may include oxygen saturation signal indicator 310, blood pressure signal indicator 320, and COx signal indicator 330. Graphical user interface 300 may include COx value indicator 340 and autoregulation status indicator 350.

Blood pressure signal indicator 320 may present a set of MAP values determined by processing circuitry 110 of regional oximetry device 100. In some examples, blood pressure signal indicator 320 may present MAP values as discrete points over time or in a table. Blood pressure signal indicator 320 may also present MAP values as a moving average or waveform of discrete points. Blood pressure signal indicator 320 may present MAP values as a single value (e.g., a number) representing a current MAP value. Oxygen saturation signal indicator 310 and COx signal indicator 330 may also present rSO₂ values and COx values, respectively, as discrete points, in a table, as a moving average, as a waveform, and/or as a single value.

In some examples, regional oximetry device 100 may be configured to determine a set of MAP values and communicate the determined set of MAP values to MPM device 150 so that both devices 100 and 150 present the same MAP values (for the same period of time). As discussed above with respect to FIG. 1, processing circuitry 110 of regional oximetry device 100 may be configured to use the same algorithm or different algorithms for determining the presented set of MAP values in blood pressure signal indicator 320 and for determining a set of MAP values to use in determining COx value indicator 340 and autoregulation status indicator 350. Additionally or alternatively, regional oximetry device 100 may determine a set of MAP values for display in blood pressure signal indicator 320 using an algorithm to mimic determination of MAP values by MPM device 150. The algorithm can be, for example, the same algorithm used by processing circuitry 160 of MPM device 150 to determine and display MAP values. In other examples, regional oximetry device 100 may also be configured to present a set of MAP values blood pressure signal indicator 320 that regional oximetry device 100 receives from MPM device 150.

These techniques for determining a set of MAP values to present via graphical user interface 300 may promote consistency between the MAP values presented as blood pressure signal indicator 320 and the set of MAP values presented by MPM device 150. These techniques may provide more consistent presentations of MAP values by devices 100 and 150, as compared to merely delivering the same raw blood pressure signal to both of devices 100 and 150. A clinician may be able to quickly determine the mean arterial pressure and autoregulation status of a patient if both of displays 120 and 170 are presenting the same MAP values. In this way, graphical user interface 300 may provide an improvement over a graphical user interface that presents MAP values that differ from the MAP values presented by MPM device 150.

Graphical user interface 300 is not the mere display of different physiological parameter values, but, rather, provides an improved graphical user interface that promotes a higher confidence in monitored physiological parameters of a patient by presenting MAP values that are consistent with MAP values presented by MPM device 150. Graphical user interface 300 may also speed up a user's determination of the most recent MAP value of a patient by presenting MAP values that are consistent with the MAP values presented by MPM device 150. Rather than choosing between different MAP values on two devices, a clinician will be presented with two consistent sets of MAP values by devices 100 and 150.

Although FIG. 3 illustrates a particular graphical user interface 300 with blood pressure signal indicator 320 displayed in addition to other information, in other examples, regional oximetry device 100, MPM device, 150, and other devices described herein may generate and present any suitable graphical user interface that includes blood pressure signal indicator 320 or other indications of determined MAP values of a patient.

FIG. 4 is a conceptual block diagram illustrating an example physiological parameter monitoring system 402. In the example shown in FIG. 4, system 402 includes sensing device 434 and regional oximetry device 400, which each generate and process physiological signals of a subject. In some examples, sensing device 434 and regional oximetry device 400 may be part of an oximeter. As shown in FIG. 4, regional oximetry device 400 includes back-end processing circuitry 414, user interface 422, light drive circuitry 430, front-end processing circuitry 450, control circuitry 470, and communication interface 490. Regional oximetry device 400 may be communicatively coupled to sensing device 434. Regional oximetry device 400 is an example of regional oximetry device 100 shown in FIG. 1 and regional oximetry device 200 shown in FIG. 2. In some examples, regional oximetry device 400 may also include a blood pressure sensor. Regional oximetry device 200, depicted in FIG. 2, is an example of a regional oximetry device that includes a blood pressure sensor.

In the example shown in FIG. 4, sensing device 434 includes light source 436, detector 438, and detector 439. Light source 436 may be configured to emit photonic signals having two or more wavelengths of light (e.g., red and IR) into a subject's tissue. For example, light source 436 may include a red light emitting light source and an IR light emitting light source, (e.g., red and IR light emitting diodes (LEDs)), for emitting light into the tissue of a subject to generate physiological signals. In some examples, the red wavelength may be between about 600 nm and about 700 nm, and the IR wavelength may be between about 800 nm and about 1000 nm. Other wavelengths of light may be used in other examples. Light source 436 may include any number of light sources with any suitable characteristics. In examples in which an array of sensors is used in place of sensing device 434, each sensing device may be configured to emit a single wavelength. For example, a first sensing device may emit only a red light while a second may emit only an IR light. In some examples, light source 436 may be configured to emit two or more wavelengths of near-infrared light (e.g., wavelengths between 600 nm and 1000 nm) into a subject's tissue. In some examples, light source 436 may be configured to emit four wavelengths of light (e.g., 724 nm, 770 nm, 810 nm, and 850 nm) into a subject's tissue.

As used herein, the term “light” may refer to energy produced by radiative sources and may include one or more of ultrasound, radio, microwave, millimeter wave, infrared, visible, ultraviolet, gamma ray or X-ray electromagnetic radiation. Light may also include any wavelength within the radio, microwave, infrared, visible, ultraviolet, or X-ray spectra, and that any suitable wavelength of electromagnetic radiation may be appropriate for use with the present techniques. Detectors 438 and 439 may be chosen to be specifically sensitive to the chosen targeted energy spectrum of light source 436.

In some examples, detectors 438 and 439 may be configured to detect the intensity of multiple wavelengths of near-infrared light. In some examples, detectors 438 and 439 may be configured to detect the intensity of light at the red and IR wavelengths. In some examples, an array of detectors may be used and each detector in the array may be configured to detect an intensity of a single wavelength. In operation, light may enter detector 438 after passing through the subject's tissue, including skin, bone, and other shallow tissue (e.g., non-cerebral tissue and shallow cerebral tissue). Light may enter detector 439 after passing through the subject's tissue, including skin, bone, other shallow tissue (e.g., non-cerebral tissue and shallow cerebral tissue), and deep tissue (e.g., deep cerebral tissue). Detectors 438 and 439 may convert the intensity of the received light into an electrical signal. The light intensity may be directly related to the absorbance and/or reflectance of light in the tissue. That is, when more light at a certain wavelength is absorbed or reflected, less light of that wavelength is received from the tissue by detectors 438 and 439.

After converting the received light to an electrical signal, detectors 438 and 439 may send the detection signals to regional oximetry device 400, where the detection signals may be processed and physiological parameters may be determined (e.g., based on the absorption of the red and IR wavelengths in the subject's tissue at both detectors). In some examples, one or more of the detection signals may be preprocessed by sensing device 434 before being transmitted to regional oximetry device 400. Additional example details of determining oxygen saturation based on light signals may be found in commonly owned U.S. Pat. No. 9,861,317, which issued on Jan. 9, 2018, and is entitled “Methods and Systems for Determining Regional Blood Oxygen Saturation,” the entire content of which is incorporated herein by reference.

Control circuitry 470 may be coupled to light drive circuitry 430, front-end processing circuitry 450, and back-end processing circuitry 414, and may be configured to control the operation of these components. In some examples, control circuitry 470 may be configured to provide timing control signals to coordinate their operation. For example, light drive circuitry 430 may generate one or more light drive signals, which may be used to turn on and off light source 436, based on the timing control signals provided by control circuitry 470. Front-end processing circuitry 450 may use the timing control signals to operate synchronously with light drive circuitry 430. For example, front-end processing circuitry 450 may synchronize the operation of an analog-to-digital converter and a demultiplexer with the light drive signal based on the timing control signals. In addition, the back-end processing circuitry 414 may use the timing control signals to coordinate its operation with front-end processing circuitry 450.

Light drive circuitry 430, as discussed above, may be configured to generate a light drive signal that is provided to light source 436 of sensing device 434. The light drive signal may, for example, control the intensity of light source 436 and the timing of when light source 436 is turned on and off. In some examples, light drive circuitry 430 provides one or more light drive signals to light source 436. Where light source 436 is configured to emit two or more wavelengths of light, the light drive signal may be configured to control the operation of each wavelength of light. The light drive signal may comprise a single signal or may comprise multiple signals (e.g., one signal for each wavelength of light).

Front-end processing circuitry 450 may perform any suitable analog conditioning of the detector signals. The conditioning performed may include any type of filtering (e.g., low pass, high pass, band pass, notch, or any other suitable filtering), amplifying, performing an operation on the received signal (e.g., taking a derivative, averaging), performing any other suitable signal conditioning (e.g., converting a current signal to a voltage signal), or any combination thereof. The conditioned analog signals may be processed by an analog-to-digital converter of circuitry 450, which may convert the conditioned analog signals into digital signals. Front-end processing circuitry 450 may operate on the analog or digital form of the detector signals to separate out different components of the signals. Front-end processing circuitry 450 may also perform any suitable digital conditioning of the detector signals, such as low pass, high pass, band pass, notch, averaging, or any other suitable filtering, amplifying, performing an operation on the signal, performing any other suitable digital conditioning, or any combination thereof. Front-end processing circuitry 450 may decrease the number of samples in the digital detector signals. In some examples, front-end processing circuitry 450 may also remove dark or ambient contributions to the received signal.

Back-end processing circuitry 414 may include processing circuitry 410 and memory 412. Processing circuitry 410 may include an assembly of analog or digital electronic components and may be configured to execute software, which may include an operating system and one or more applications, as part of performing the functions described herein. Processing circuitry 410 may receive and further process physiological signals received from front-end processing circuitry 450. For example, processing circuitry 410 may determine one or more physiological parameter values based on the received physiological signals. For example, processing circuitry 410 may compute one or more of regional oxygen saturation, blood oxygen saturation (e.g., arterial, venous, or both), pulse rate, respiration rate, respiration effort, blood pressure, hemoglobin concentration (e.g., oxygenated, deoxygenated, and/or total), any other suitable physiological parameters, or any combination thereof. Processing circuitry 410 may perform any suitable signal processing of a signal, such as any suitable band-pass filtering, adaptive filtering, closed-loop filtering, any other suitable filtering, and/or any combination thereof. Processing circuitry 410 may also receive input signals from additional sources not shown. For example, processing circuitry 410 may receive an input signal containing information about treatments provided to the subject from user interface 422. Additional input signals may be used by processing circuitry 410 in any of the determinations or operations it performs in accordance with back-end processing circuitry 414 or regional oximetry device 400.

Memory 412 may include any suitable computer-readable media capable of storing information that can be interpreted by processing circuitry 410. In some examples, memory 412 may store reference absorption curves, reference sets, determined values, such as blood oxygen saturation, pulse rate, blood pressure, fiducial point locations or characteristics, initialization parameters, any other determined values, or any combination thereof, in a memory device for later retrieval. Back-end processing circuitry 414 may be communicatively coupled with user interface 422 and communication interface 490.

User interface 422 may include user input 424, display 420, and speaker 426. User interface 422 may include, for example, any suitable device such as one or more medical devices (e.g., a medical monitor that displays various physiological parameters, a medical alarm, or any other suitable medical device that either displays physiological parameters or uses the output of back-end processing 414 as an input), one or more display devices (e.g., monitor, personal digital assistant (PDA), mobile phone, tablet computer, clinician workstation, any other suitable display device, or any combination thereof), one or more audio devices, one or more memory devices, one or more printing devices, any other suitable output device, or any combination thereof.

User input 424 may include any type of user input device such as a keyboard, a mouse, a touch screen, buttons, switches, a microphone, a joy stick, a touch pad, or any other suitable input device or combination of input devices. User input 424 may also receive inputs to select a model number of sensing device 434, blood pressure sensor 232 (FIG. 2), or blood pressure processing equipment. In some examples, processing circuitry 410 may associate each model number of sensing device and processing equipment with a known time delay in memory 412, and use the known time delay of sensing device to determine a set of MAP values for determining COx values and an autoregulation status.

In some examples, the subject may be a medical patient and display 420 may exhibit a list of values which may generally apply to the subject, such as, for example, an oxygen saturation signal indicator, a blood pressure signal indicator, a COx signal indicator, a COx value indicator, and/or an autoregulation status indicator. Display 420 may also be configured to present additional physiological parameter information. Graphical user interface 300 is an example of an interface that can be presented via display 420 of FIG. 4. Additionally, display 420 may display, for example, one or more estimates of a subject's regional oxygen saturation generated by regional oximetry device 400 (referred to as an “rSO₂” measurement). Speaker 426 within user interface 422 may provide an audible sound that may be used in various examples, such as for example, sounding an audible alarm in the event that a patient's physiological parameters are not within a predefined normal range.

Communication interface 490 may enable regional oximetry device 400 to exchange information with external devices. Communication interface 490 may include any suitable hardware, software, or both, which may allow regional oximetry device 400 to communicate with electronic circuitry, a device, a network, a server or other workstations, a display, or any combination thereof. For example, regional oximetry device 400 may communicate MAP values to MPM device 150 (FIG. 1) via communication interface 490 or regional oximetry device 400 may receive MAP values from MPM device 150 via communication interface 490.

The components of physiological monitoring system 402 that are shown and described as separate components are shown and described as such for illustrative purposes only. In some examples the functionality of some of the components may be combined in a single component. For example, the functionality of front end processing circuitry 450 and back-end processing circuitry 414 may be combined in a single processor system. Additionally, in some examples the functionality of some of the components of regional oximetry device 400 shown and described herein may be divided over multiple components. For example, some or all of the functionality of control circuitry 470 may be performed in front end processing circuitry 450, in back-end processing circuitry 414, or both. In other examples, the functionality of one or more of the components may be performed in a different order or may not be required. In some examples, all of the components of physiological monitoring system 402 can be realized in processor circuitry.

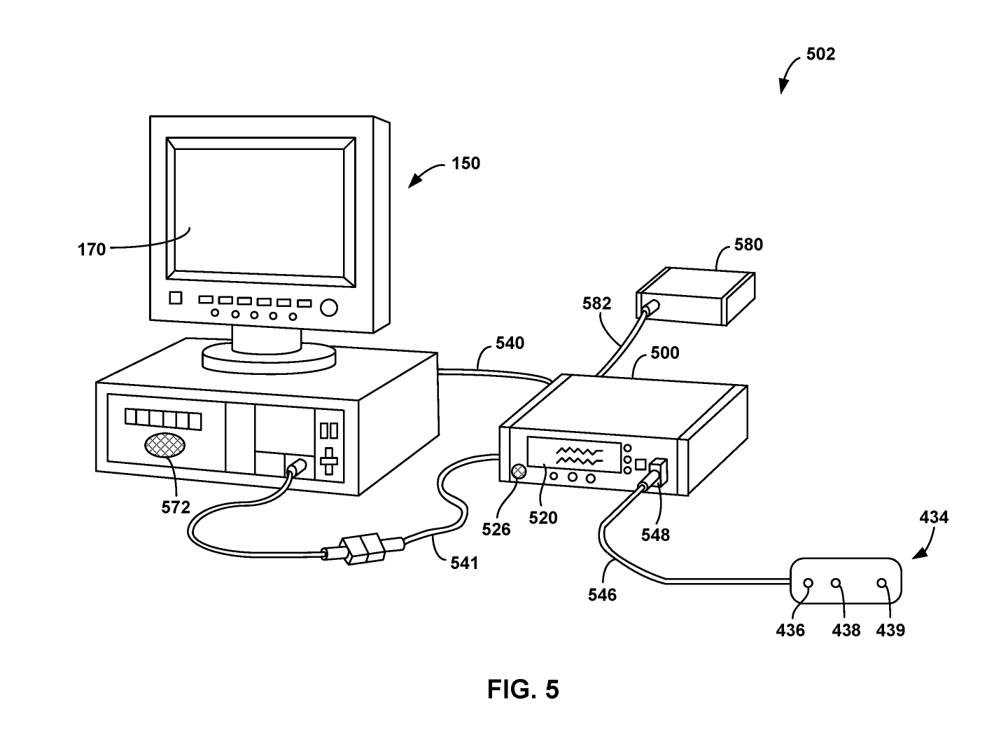

FIG. 5 is a perspective-view diagram illustrating an example physiological parameter monitoring system 502. Physiological parameter monitoring system 502 is an example of the system shown in FIG. 1, which includes regional oximetry device 100 and MPM device 150. Physiological monitoring system 502 may include sensing device 434 and regional oximetry device 500, where regional oximetry device 500 is an example of regional oximetry device 100 shown in FIG. 1. In some examples, sensing device 434 may be part of an oximeter and may include one or more light sources 436 for emitting light at one or more wavelengths into a subject's tissue. Physiological monitoring system 502 may also include one or more additional sensor units (not shown) that may, for example, take the form of any of the examples described herein with reference to sensing device 434. An additional sensor unit may be the same type of sensor unit as sensing device 434, or a different sensor unit type than sensing device 434 (e.g., a photoacoustic sensor). Multiple sensor units may be capable of being positioned at two different locations on a subject's body.

In some examples, sensing device 434 may be electrically connected to regional oximetry device 500 as shown via a wired connection (as shown) or via a wireless connection. Sensing device 434 may be powered by an internal power source, e.g., a battery (not shown), or may draw power from regional oximetry device 500.

Regional oximetry device 500 may be configured to determine physiological parameter values of a patient based at least in part on data relating to light emission and acoustic detection received from one or more sensor units such as sensing device 434. For example, regional oximetry device 500 may be configured to determine regional oxygen saturation, pulse rate, respiration rate, respiration effort, blood pressure, blood oxygen saturation (e.g., arterial, venous, or both), hemoglobin concentration (e.g., oxygenated, deoxygenated, and/or total), any other suitable physiological parameters, or any combination thereof. In some examples, determinations may be performed on the sensor units or an intermediate device and the result of the determinations may be passed to regional oximetry device 500.

Regional oximetry device 500 may include display 520 configured to display the physiological parameters or other information about the system. In the example shown in FIG. 5, regional oximetry device 500 may also include a speaker 526 to provide an audible sound that may be used in various other examples, such as for example, sounding an audible alarm in the event that a subject's physiological parameters are not within a predefined normal range. In some examples, physiological monitoring system 502 may include a stand-alone monitor in communication with the regional oximetry device 500 via a cable or a wireless network link.

In some examples, sensing device 434 may be communicatively coupled to regional oximetry device 500 via a cable 546 at port 548. Cable 546 may include electronic conductors (e.g., wires for transmitting electronic signals from detectors 438 and 439), optical fibers (e.g., multi-mode or single-mode fibers for transmitting emitted light from light source 436), any other suitable components, any suitable insulation or sheathing, or any combination thereof. In some examples, a wireless transmission device (not shown) or the like may be used instead of or in addition to cable 546. Regional oximetry device 500 may include a sensor interface configured to receive physiological signals from sensing device 434, provide signals and power to sensing device 434, or otherwise communicate with sensing device 434. The sensor interface may include any suitable hardware, software, or both, which may allow communication between regional oximetry device 500 and sensing device 434.

In some examples, physiological monitoring system 502 may include calibration device 580. In some examples, calibration device 580 may include a manual input device (not shown) used by an operator to manually input reference signal measurements obtained from some other source (e.g., an external invasive or non-invasive physiological measurement system). Calibration device 580, which may have any suitable power source (e.g., regional oximetry device 500, a battery, or a conventional power source such as a wall outlet), may include any suitable calibration device. Calibration device 580 may be communicatively coupled to regional oximetry device 500 via communicative coupling 582, and/or may communicate wirelessly (not shown). In some examples, calibration device 580 is completely integrated within regional oximetry device 500.

MPM device 150 may include any suitable display device 170, such as, but not limited to, a cathode ray tube display, a flat panel display (as shown) such as a liquid crystal display (LCD) or a plasma display, or may include any other type of monitor now known or later developed. MPM device 150 may be configured to determine physiological parameters and to present, via display 170, information from regional oximetry device 500 and from other medical monitoring devices or systems (not shown). For example, MPM device 150 may be configured to display an estimate of a subject's blood oxygen saturation and hemoglobin concentration generated by regional oximetry device 500. MPM device 150 may include a speaker 572.

Regional oximetry device 500 may be communicatively coupled to MPM device 150 via a cable 540 or 541 that is coupled to a sensor input port or a digital communications port, respectively and/or may communicate wirelessly (not shown). In addition, regional oximetry device 500 and/or MPM device 150 may be coupled to a network to enable the sharing of information with servers or other workstations (not shown).

Devices 500 and 150 may be configured to communicate MAP values with each other (one way or both ways) via cables 540 and/or 541. For example, regional oximetry device 500 may be configured to determine a set of MAP values to present via display 520. Regional oximetry device 500 may communicate the set of MAP values to MPM device 150 so that devices 500 and 150 present the same MAP values via the respective displays 520 and 170. Communication of MAP values between devices 500, 150 may improve the operation of devices 500 and 150 by increasing the consistency between the MAP values presented via displays 520 and 170. Consistency between the presented MAP values may increase the confidence that a user has in the data presented via displays 520 and 170.

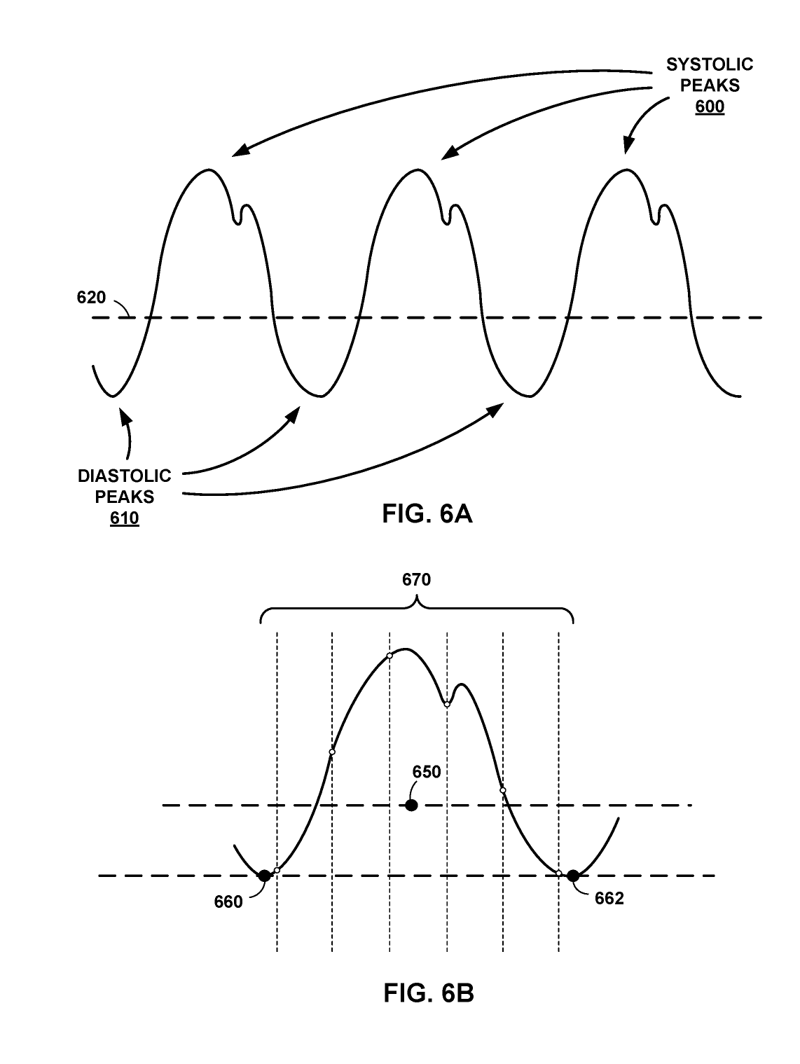

Regional oximetry devices (e.g., devices 100, 200, 400, and/or 500) and MPM device 150 may determine MAP values using any suitable technique. The regional oximetry device and/or MPM device 150 may be configured to determine MAP values based on a blood pressure signal received from a blood pressure sensor, such as sensing device 132 and/or 182 (FIG. 1). The blood pressure signal may include a waveform (See FIGS. 6A and 6B) that includes systolic peaks and diastolic peaks (or troughs).

FIGS. 6A and 6B illustrate example blood-pressure waveforms. Regional oximetry device 100 (e.g., processing circuitry 110) shown in FIG. 1 may determine a MAP value based on systolic peaks 600 and diastolic peaks 610. In accordance with a first algorithm, regional oximetry device 100 may determine an average of the amplitudes of one or more of systolic peaks 600 and one or more of diastolic peaks 610, as shown in Equation (1). In accordance with a second algorithm, regional oximetry device 100 may determine a weighted average as shown in Equation (2), using a weighting of k equals two in favor of diastolic peaks 610, for example. A value of k that is greater than one, means that a weighting of diastolic peaks 610 is greater than a weighting of systolic peaks 600. FIG. 6A depicts average amplitude 620 as an example of a MAP value determined using the second algorithm.

$$\begin{matrix}MAP value = \frac{1}{2 N} [ {\sum }_{i = 1}^{N} ( {BP}_{{systolic}_{i}} + {BP}_{{diastolic}_{i}} ) & ( 1 ) \\ MAP value = \frac{1}{N ( k + 1 )} {\sum }_{i = 1}^{N} {BP}_{{systolic}_{i}} + \frac{k}{N ( k + 1 )} {\sum }_{i = 1}^{N} {BP}_{{diastolic}_{i}} & ( 2 ) \\ MAP value = \frac{1}{N} {\sum }_{i = 1}^{N} {BP}_{i} & ( 3 )\end{matrix}$$

In accordance with a third algorithm, regional oximetry device 100 may determine a MAP value using the centroid of the waveform. Regional oximetry device 100 may determine the centroid by sampling the blood pressure waveform at a relatively high rate (e.g., one hundred times the frequency of the waveform) and averaging all of the sampled amplitudes, as shown in Equation (3). FIG. 6B shows an example of determining a MAP value using centroid 650, which may include sampling along the blood pressure waveform between diastolic peak 660 and diastolic peak 662. For example, FIG. 6B depicts six sampling points 670 between the times of diastolic peak 660 and diastolic peak 662. In accordance with the third algorithm, regional oximetry device 100 may determine an average of an ensemble pulse waveform by averaging all of the sampled amplitudes.

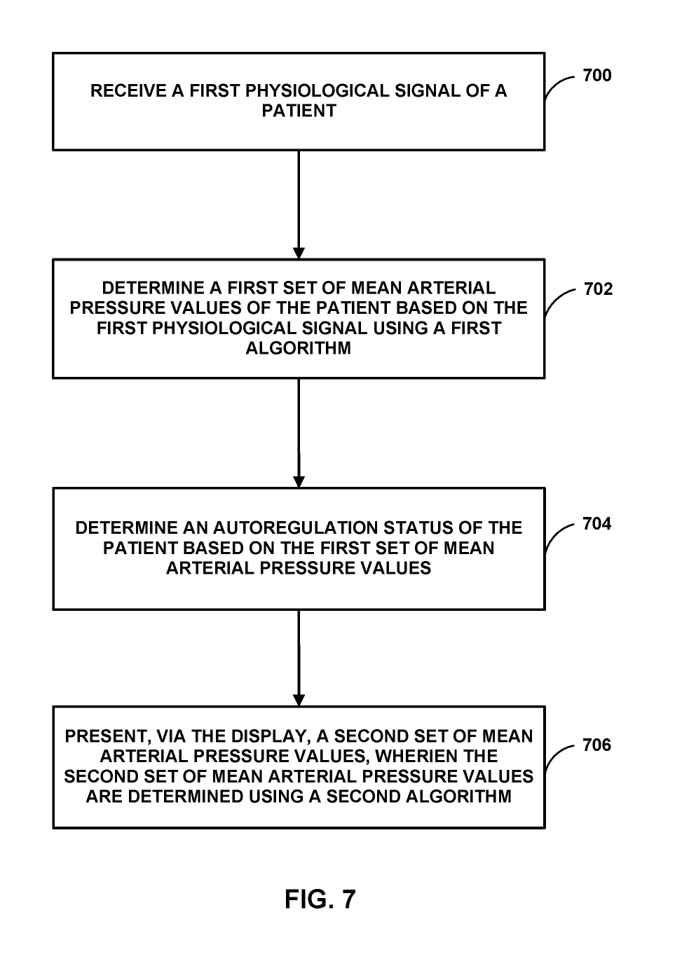

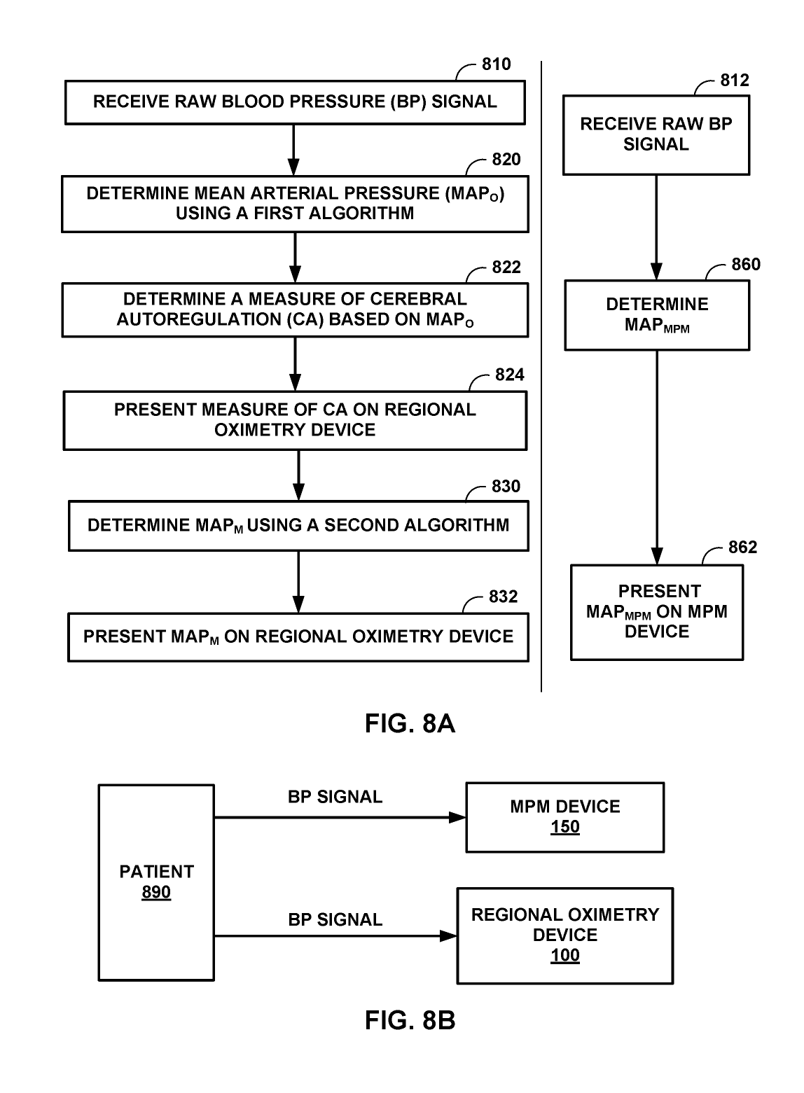

Regional oximetry device 100 may be configured to use one or more of the algorithms described above to determine a first set of MAP values for determining an autoregulation status of a patient. Additionally or alternatively, regional oximetry device 100 may be configured to use one or more of the algorithms to determine a second set of set of MAP values for presenting via display 120. In some examples, regional oximetry device 100 may be configured to use one or more of the algorithms to mimic the determination of a set of MAP values by MPM device 150. These algorithms may be used to determine MAP values for transmitting to MPM device 150 (see FIG. 7) and/or for determining an autoregulation status of a patient (see FIG. 8).

FIG. 7 is a flow diagram illustrating example techniques for presenting coherent MAP values, in accordance with some examples of this disclosure. Although FIG. 7 are described with respect to processing circuitry 110 of regional oximetry device 100 (FIG. 1), in other examples, processing circuitry 160, 210, 410, 414, and/or 450, alone or in combination with processing circuitry 110, may perform any part of the techniques of FIG. 7.

In the example techniques of FIG. 7, processing circuitry 110 receives a first physiological signal of a patient (700). Processing circuitry 110 may receive the first physiological signal directly from a sensing device, or from another processing device (e.g., processing circuitry 160 of MPM device 150). The first physiological signal may be, for example, a blood pressure signal received from sensing device 132 and sensing circuitry 130. Sensing device 132 may include a blood pressure cuff and/or a pulse oximetry sensor configured to receive a PPG signal.

Processing circuitry 110 determines a first set of mean arterial pressure (MAP) values of the patient for a time period based on the first physiological signal using a first algorithm (702). The first algorithm can be any suitable algorithm for calculating MAP values from a blood pressure signal, such as, but not limited to, Equations (1), (2), and/or (3) described with respect to FIGS. 6A and 6B above. In some examples, processing circuitry 110 may use an algorithm that is specifically tailored to patients undergoing a cardiovascular medical procedure or to neo-natal patients.

Processing circuitry 110 determines an autoregulation status of the patient based on the first set of MAP values (704). Processing circuitry 110 may be configured to determine the autoregulation status by determining a set of COx values based on the first set of MAP values and a set of oxygen saturation values. Processing circuitry 110 may receive the set of oxygen saturation values for the time period from, for example, sensing device 132 and sensing circuitry 130 (e.g., oxygen saturation sensor 234 shown in FIG. 2). In some examples, processing circuitry 110 determines a lower limit of autoregulation and an upper limit of autoregulation in terms of MAP in order to determine whether the most current MAP value determined for the patient is between or outside of the limits of autoregulation. In particular, processing circuitry 110 may be configured to determine an autoregulation status based on whether one or more MAP values are less than a lower limit of autoregulation.

Processing circuitry 110 presents, via display 120, a second set of MAP values of the patient for the time period, wherein the second set of mean arterial pressure values are determined using a second algorithm (706). In some examples, the second algorithm may be the same as the first algorithm, such that processing circuitry 110 determines only one set of MAP values for presentation and determination of autoregulation status. In other examples, however, the first and second algorithms for determining MAP values are different, such that processing circuitry 110 determines the autoregulation status of the patient (704) based on a first set of MAP values that differs from the second set of MAP values that processing circuitry 110 presents to a user via display 120.

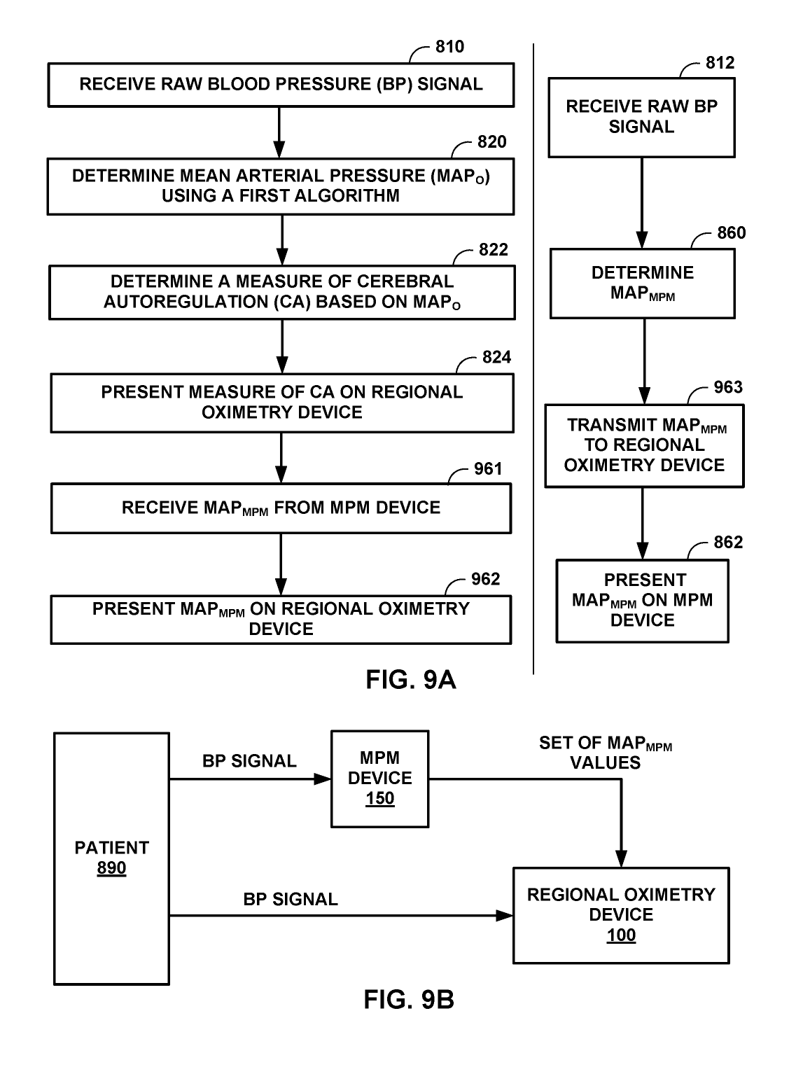

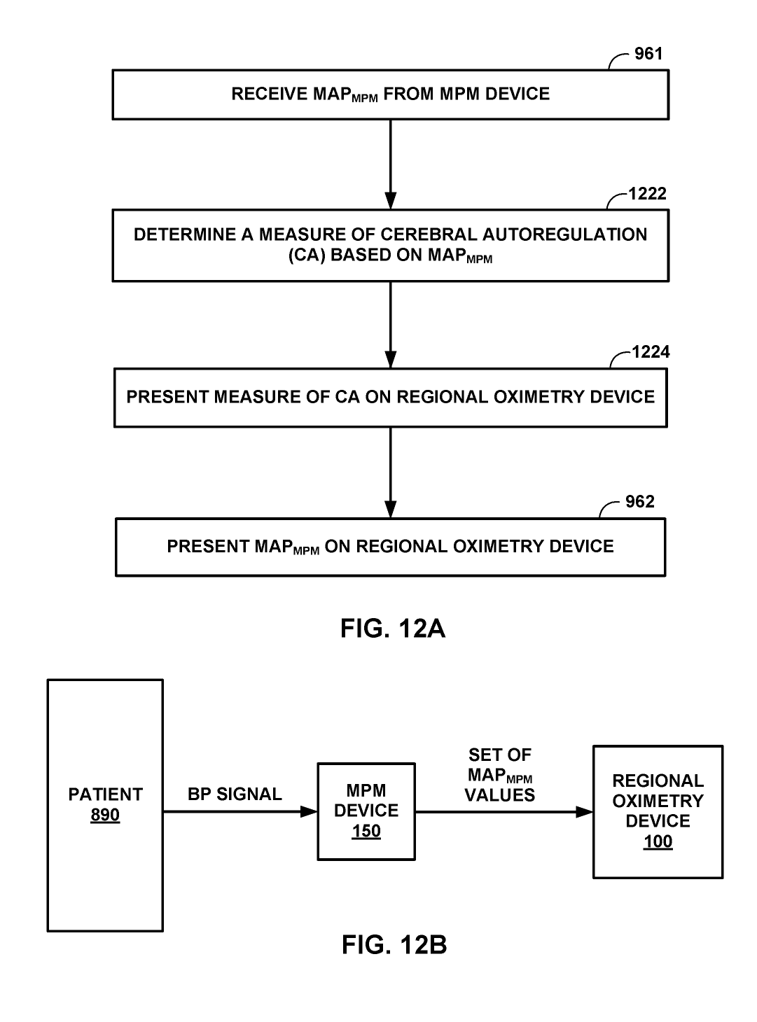

Processing circuitry 110 determines the second set of MAP values using any suitable technique. In some examples, processing circuitry 110 receives the second set of MAP values from another device, such as MPM device 150. In other examples, processing circuitry 110 is configured to determine two distinct sets of MAP values for the time period, the first set for determination of autoregulation status and the second set for presentation. In some of these examples, processing circuitry 110 does not present the first set of MAP values via display 120 and only presents the second set of MAP values via display 120. Processing circuitry 110 may determine the second set of MAP values to mimic the determination of MAP values by MPM device 150 (or another patient monitoring device) or processing circuitry 110 may determine the second set of MAP values and transmit the second set of MAP values to MPM device 150 for presentation via display 170. Additionally or alternatively, processing circuitry 110 may also be configured to receive the first set of MAP values and/or the second set of MAP values from another device, e.g., MPM device 150 as described with respect to, for example, FIGS. 9A, 9B, 12A, and 12B.

FIGS. 8A-12B are flow diagrams of several possible examples for coordinating the MAP values that are presented by regional oximetry device 100 and by MPM device 150. The examples of FIGS. 8A-12B show example techniques for improving the consistency between the MAP values that are presented by the two devices, thereby improving the operation of one of the particular devices and/or the system including devices 100, 150. The examples of FIGS. 8A-12B may also be modified to include delivering and presenting consistent sets of values on both displays for other physiological parameters, such as blood volume saturation, hemoglobin volume index, and the like.

FIGS. 8A and 8B are a flow diagram and a conceptual block diagram, respectively, illustrating an example technique performed by regional oximetry device 100 to determine and display MAP to mimic the MAP determined by an MPM device 150, as well as a corresponding example technique performed by MPM device 150. In the example shown in FIG. 8A, both devices 100 and 150 may receive one or more raw blood pressure signals from patient 890, from which MAP values may be determined (810, 812). Devices 100 and 150 may receive the raw blood pressure signals from any suitable device, such as, for example, one or both of sensing devices 132 and 182. In some examples, one of sensing devices 132 or 182 may be configured to deliver the same blood pressure signal to both of devices 100 and 150. Additionally or alternatively, sensing device 132 may provide a first blood pressure signal to processing circuitry 110 of regional oximetry device 100, and sensing device 182 may provide a second blood pressure signal to processing circuitry 160 of MPM device 150.

In the example shown in FIGS. 8A and 8B, each of devices 100 and 150 use different algorithms to determine MAP values to present via respective displays 120, 170. Processing circuitry 110 of regional oximetry device 100 may determine a first set of MAP values (MAP_{O}) using a first algorithm (820) and may determine a measure of CA based on the first set of MAP values (822), e.g., as described with respect to FIG. 7. Regional oximetry device 100 may present the measure of CA via display 120 (824). In some examples, processing circuitry 110 presents the measure of CA as the autoregulation status of patient 890, indicated by red or green color, or a COx value, and/or as the lower limit of autoregulation or the upper limit of autoregulation, as expressed in units of MAP, such as mmHg (millimeters of mercury) or kilopascals (kPa).

Processing circuitry 110 may also determine a second set of MAP values (MPM_{M}) using a second algorithm (830) and present the second set of MAP values via display 120 (832). In the example shown in FIGS. 8A and 8B, the second algorithm is different from the first algorithm and results in a different set of MAP values, even though the MAP values are determined using the same underlying blood pressure signal (810) for the same time period. Using the second algorithm, processing circuitry 110 may mimic the determination of MAP by MPM device 150. Processing circuitry 110 may be configured to use a default algorithm to mimic the determination of MAP by MPM device 150, or a user may input (via a user interface of regional oximetry device 100) the model number of MPM device 150, and processing circuitry 110 100 may select the second algorithm to mimic the algorithm used by MPM device 150 based on the received model number. Processing circuitry 110 may determine the first and second sets of MAP values at the same time or at different times. For example, processing circuitry 110 may determine the first and second sets of MAP values in series (e.g., one after the other) or in parallel (e.g., with two or more processors or threads operating in parallel).

As shown in FIG. 8A, during the same time as or at a different time as the determination of the first and second sets of MAP values (820, 830) by processing circuitry 110, processing circuitry 160 of MPM device 150 may receive a raw blood pressure signal (812) and determine a third set of MAP values (MPM_{MPM}) based on the raw blood pressure signal (860). Processing circuitry 160 may present the third set of MAP values via display 170 (862). The third set of MAP values may be the same as or similar to the second set of MAP values because processing circuitry 110 and 160 may use the same or similar algorithms for determining the second and third sets of MAP values. Regional oximetry device 100 may use a second algorithm that is different from the first algorithm in order to improve the consistency between the MAP values presented by devices 100 and 150.

In some examples, processing circuitry 110 is configured to determine a signal quality metric (Q) based on a difference between the first set of MAP values (MAP_{O}) and the second set of MAP values (MAP_{M}), as shown in Equation (4). In Equation (4), Q is equal to a difference between an average of N number of MAP_{O} values and an average of K number of MAP_{M} values.

$$Q = \frac{1}{N} {\sum }_{i = 1}^{N} {MAP}_{{O}_{i}} - \frac{1}{K} {\sum }_{i = 1}^{K} {MAP}_{{M}_{i}} \qquad ( 4 )$$

A high value of the signal quality metric may indicate that the first set of MAP values is diverging from the second set of MAP values, possibly due to issues (e.g., noise or an improper interface between a blood pressure measuring device and patient 890) with the received blood pressure signal. A high value of the signal quality metric may also indicate a delay between the blood pressure signal and the oxygen saturation signal, an artifact in the blood pressure signal (as discussed above), e.g., due to movement of a sensing device or the patient, and/or flushing of the arterial line. If processing circuitry 110 determines that the signal quality metric is greater than or equal to a threshold level, then regional oximetry device 100 may generate the notification (e.g., an alert) via display 120 or another user interface mechanism. The threshold level may be, for example, five, ten, or fifteen mmHg. In some examples, processing circuitry 110 presents the notification via display 120 as a color (e.g., red) or a textual description of the signal quality metric value exceeding the threshold level. Regional oximetry device 100 may also refrain from outputting the measure of CA, which may include the autoregulation status, in response to determining that the signal quality metric is greater than or equal to the threshold level.

FIGS. 9A and 9B are a flow diagram and a conceptual block diagram, respectively, illustrating an example technique performed by regional oximetry device 100 to receive and display MAP values determined by an MPM device 150, as well as a corresponding example technique performed by MPM device 150. In the example of FIGS. 9A and 9B, both of processing circuitry 110 and 160 are configured to present the same set of MAP values (MAP_{MPM}). Processing circuitry 160 receives a raw blood pressure signal (812), determines the set of MAP values (860), and communicates the set to regional oximetry device 100 (963).

Processing circuitry 110 and 160 may be configured to receive the same blood pressure signal from one of sensing devices 132 and 182, or processing circuitry 110 and 160 may be configured to receive different blood pressure signals from sensing devices 132 and 182. Processing circuitry 110 may also determine a first set of MAP values (MAP_{O}) using a first algorithm (820). The first algorithm may be specifically tailored to the unique pulse morphologies that occur in particular patient groups, such as cardiovascular-surgery patients and neo-natal subjects. In the example shown in FIGS. 9A and 9B, the second algorithm is different from the first algorithm and results in a different set of MAP values, even though processing circuitry 110 and 160 may determine the sets of MAP values using the same underlying blood pressure signal for the same time period. Processing circuitry 110 may determine a measure of CA and present the measure of CA based on the first set of MAP values (822, 824).

In the example shown in FIGS. 9A and 9B, processing circuitry 110 is configured to present the set of MAP_{MPM} values received from MPM device 150 (961, 962) and determine another set of MAP values (MAP_{O}) for determination the measure of CA (820). Processing circuitry 110 may present the set of MAP values of MAP_{MPM} instead of or in addition to the set of MAP_{O} values determined using the first algorithm in order to present MAP values that are consistent with those presented by processing circuitry 160 of MPM device 150 (862). However, because processing circuitry 110 uses the set of MAP_{O} values (determined using an algorithm that is specifically tailored for cardiovascular medical procedures, neo-natal monitoring, or another patient population) to determine the CA status of the patient, processing circuitry 110 may determine a more accurate CA status of patient compared to if the set of MAP_{MPM} values received from MPM device 150 was used to determine the CA status. In this way, the function of processing circuitry 110 may be improved, and processing circuitry 110 may be configured to present an improved graphical user interface that includes MAP_{MPM} values received from MPM device 150. The graphical user interface may be improved because, for example, the MAP values presented via display 120 of regional oximetry device 100 match or substantially match (within a particular range, such as 5% of MAP values) the MAP values presented by display 170 of MPM device 150, thereby improving clinician confidence in the displayed MAP values.

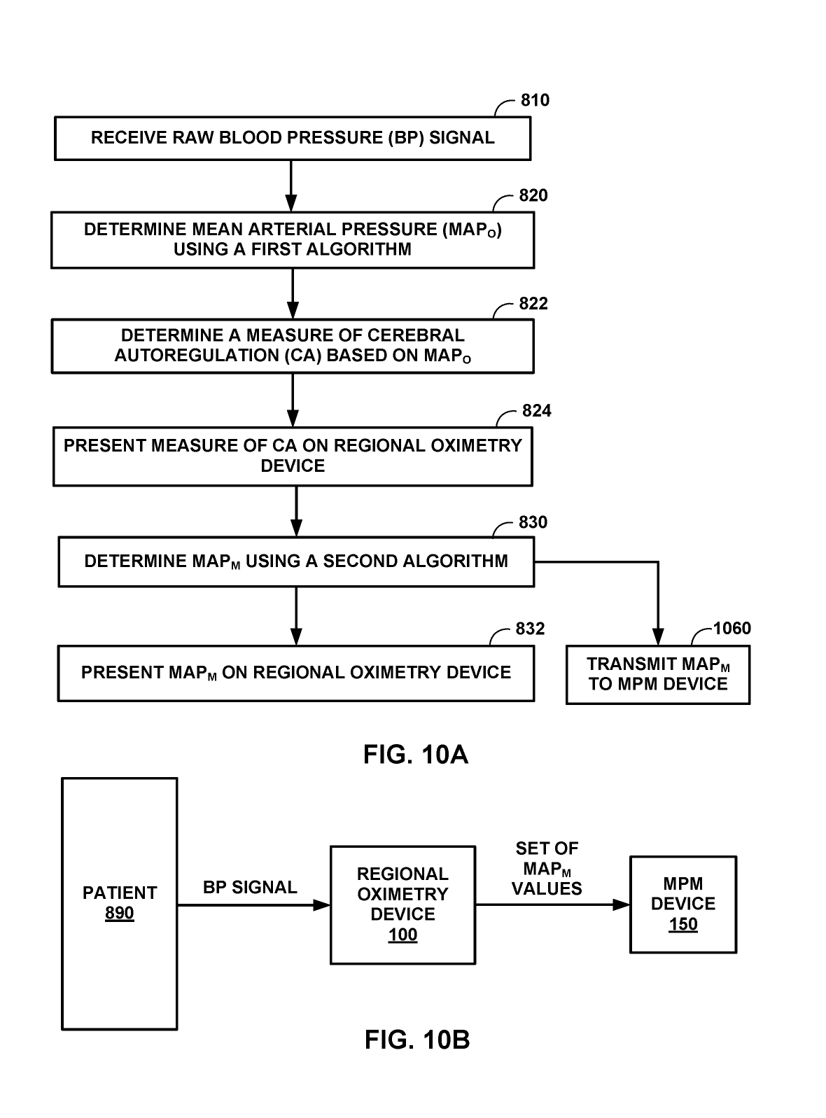

FIGS. 10A and 10B are a flow diagram and a conceptual block diagrams illustrating an example technique performed by regional oximetry device 100 to determine, display, and communicate MAP values to an MPM device 150. In the example of FIG. 10A, processing circuitry 110 is configured to receive a blood pressure signal from patient 890 via sensing device 132 (810) and determine a first set of MAP values using a first algorithm (820) based on the raw blood pressure signal. Processing circuitry 110 may also be configured to determine a second set of MAP values (MAP_{M}) using a second algorithm (830) based on the raw blood pressure signal. In the example shown in FIGS. 8A and 8B, the second algorithm is different from the first algorithm and results in a different set of MAP values, even though the sets of MAP values are determined using the same underlying blood pressure signal for the same time period. Processing circuitry 110 may communicate (e.g., transmit) the second set of MAP values to MPM device 150 for presentation on display 170 (1060).

In the example of FIGS. 10A and 10B, processing circuitry 110 and 160 may present the same set of MAP values because the MAP values presented by MPM device 150 are received from regional oximetry device 100. As with the previous examples, the coherence between the MAP values displayed by regional oximetry device 100 and MPM device 150 may result in an improved graphical user interface, thereby improving clinician confidence in the displayed MAP values.

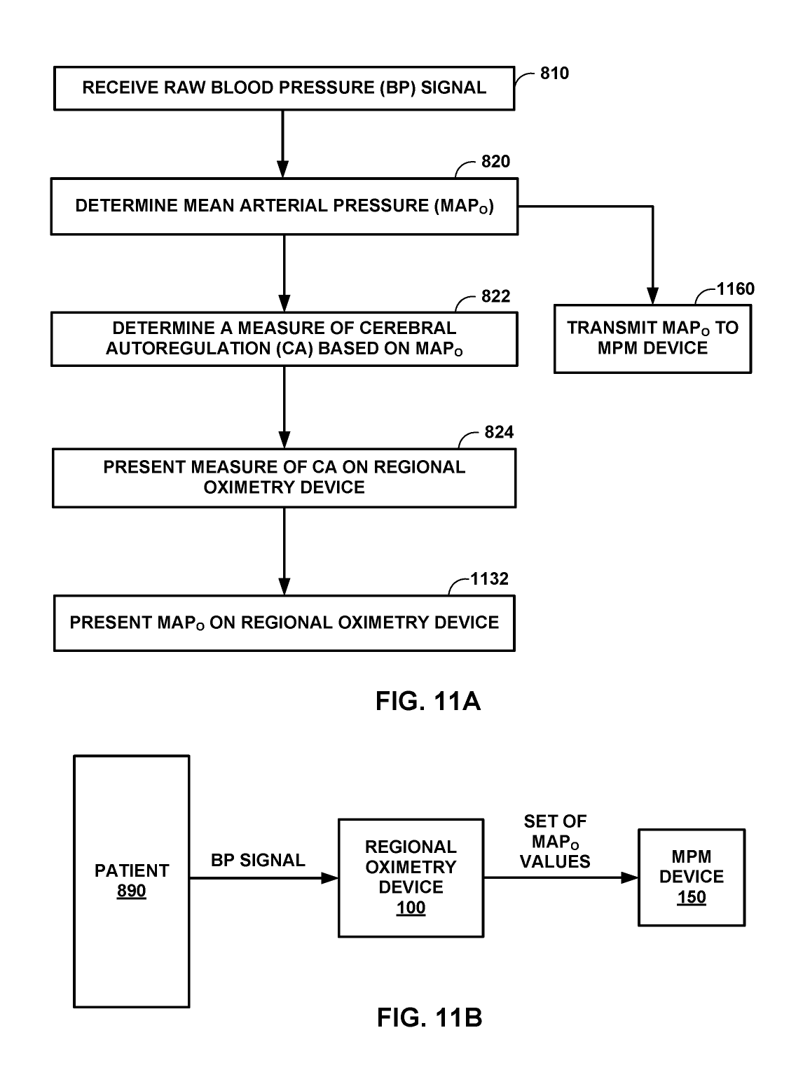

FIGS. 11A and 11B are a flow diagram and a conceptual block diagram illustrating an example technique performed by regional oximetry device 100 to determine, display, and communicate MAP values to an MPM device 150 using one algorithm. In the example of FIG. 11A, processing circuitry 110 receives a blood pressure signal from patient 890 via sensing device 132 (810) and determines a set of MAP values (MAP_{O}) (820) and uses the set of MAP values for three purposes. First, processing circuitry 110 determines and presents a measure of CA (822, 824). Second, processing circuitry 110 presents the determined set of MAP values (1132). Third, processing circuitry 110 communicates the set of MAP values to MPM device 150 for presentation by MPM device 150 (1160).

FIGS. 12A and 12B are a flow diagram and a conceptual block diagram illustrating an example technique performed by regional oximetry device 100 to receive and display MAP values determined by an MPM device 150. In the example of FIG. 12A, processing circuitry 160 receives a raw blood pressure signal from sensing device 182 and determines a set of MAP values (MAP_{MPM}). In the example of FIG. 12A, processing circuitry 110 and 160 may use the set of MAP values for three purposes. First, processing circuitry 160 presents the set of MAP values via a display. Second, processing circuitry 160 communicates the set of MAP values to regional oximetry device 100, and processing circuitry 110 of regional oximetry device 100 receives the set of MAP values (1221) and presents the received set of MAP values via a display (962). Third, processing circuitry 110 also uses the received set of MAP values to determine and present a measure of CA (1232).

In order to determine a measure of CA, processing circuitry 110 may determine a first set of MAP values. Processing circuitry 110 may also determine a set of oxygen saturation values based on a NIRS signal received from sensing devices 132. Processing circuitry 110 may then be configured to determine a set of COx values based on the first set of MAP values and the set of oxygen saturation values. Processing circuitry 110 may determine the measure of CA (e.g., the most recent COx value, a limit of autoregulation, or an autoregulation status) based on the set of COx values.

In some examples, to improve the accuracy of the measure of CA, processing circuitry 110 may be configured to synchronize, in time, the first set of MAP values and the set of oxygen saturation values. Processing circuitry 110 may then be configured to determine the measure of CA based on the synchronized set of MAP values and the synchronized set of oxygen saturation values. Using the synchronized data sets may result in a more accurate determination of the measure of CA because the MAP values and oxygen saturation values may be indicative of the same patient state, rather than patient states at different times from each other. Thus, processing circuitry 110 may present more accurate information about the autoregulation status of a patient to a clinician, allowing the clinician to take appropriate actions.

FIG. 13 is a flow diagram illustrating an example technique for synchronizing MAP values and oxygen saturation values. Although FIG. 13 refers to processing circuitry 210 of FIG. 2, in other examples, processing circuitry 110, 160, 410, 414, and/or 450, alone or in combination with processing circuitry 210, may perform any part of the technique of FIG. 13. In the example technique of FIG. 13, processing circuitry 210 receives a first physiological signal of a patient and a second physiological signal of the patient (1300). For example, blood pressure sensor 232 may generate a first signal indicating a blood pressure of the patient, and oxygen saturation sensor 234 may generate a signal indicating an oxygen saturation of the patient. Sensors 232 and 234 may deliver the physiological signals directly to processing circuitry 210, and/or one or both of sensors 232 and 234 may perform pre-processing on the physiological signals.

Processing circuitry 210 determines a set of MAP values of the patient based on the first physiological signal (1302). Processing circuitry 210 may determine the set of MAP values using any suitable algorithm, such as, but not limited to, Equations (1), (2), and/or (3) described with respect to FIGS. 6A and 6B above. Processing circuitry 210 also determines a set of oxygen saturation values of the patient based on the second physiological signal (1304). Processing circuitry 210 may determine the set of oxygen saturation values using any suitable technique, such as one or more of the techniques described with respect to FIG. 4. The set of MAP values and the set of oxygen saturation values may not necessarily be synchronized in time.

Processing circuitry 210 determines a relative time delay between the set of oxygen saturation values and the set of MAP values (1306). In some examples, processing circuitry 210 may determine the relative time delay based on a force applied to both of sensors 232 and 234. For example, processing circuitry 210, via output device 222, may direct a user to flick or tap (e.g., with a finger) the probes of both of sensors 232 and 234 at the same time. Processing circuitry 210 may detect the spike in each of the signals received from sensors 232 and 234 and determine the time lag between the spike in each signal, where the time lag may be equal to the time delay. The time lag may be zero if there is no relative time delay between the two signals. Processing circuitry 210 may cause output device 222 to direct the user to perform this test several times to determine an average of time delays for each test. Another testing technique may include inputting dummy signals into sensors 232 and 234 and having processing circuitry 210 measure the relative time delay in receiving the dummy signals.

These tests may provide the relative time delay for processing circuitry 210 receiving the signals (e.g., an upstream delay). In addition to or instead of the techniques described above, processing circuitry 210 may determine an additional relative time delay (e.g., a downstream delay) based on the processing of the signals to produce a set of MAP values and a set of oxygen saturation values. For example, if processing circuitry 210 uses sampling windows (e.g., analysis windows) with different characteristics such as different durations, the different characteristics may cause an additional relative time delay between the set of MAP values and the set of oxygen saturation values.

Processing circuitry 210 determines an autoregulation status of the patient based on the set of MAP values, the set of oxygen saturation values, and the relative time delay between the first physiological signal and the second physiological signal (1308). Processing circuitry 210 may be configured to synchronize the data sets using the relative time delay by, for example, adding or subtracting the determined delay from the time (e.g., timestamp) associated with each value in the set. For example, processing circuitry 210 may store a multi-dimensional data structure such as an array to memory 212, where the data structure associates each MAP value with a time of measurement. Processing circuitry 110 may adjust (e.g., shift) the associated time by the determined delay in order to synchronize the set of MAP values with the set of oxygen saturation values. Processing circuitry 210 may then determine a set of COx values based on the synchronized set of MAP values and the synchronized set of oxygen saturation values (1308). For example, processing circuitry 210 may determine the limits of autoregulation based on the set of COx values and then determine the autoregulation status based on whether the current MAP value is between or outside of the limits of autoregulation.

By determining the relative time delay and using the relative time delay to determine the autoregulation status, processing circuitry 210 may improve the accuracy of the determination of the autoregulation status, as compared to another regional oximetry device that uses unsynchronized data. For example, another regional oximetry device that uses unsynchronized data may determine incorrect limits of autoregulation and then incorrectly determine that the patient has intact autoregulation or impaired autoregulation. Thus, by using the relative time delay to determine the autoregulation status, processing circuitry 210 may reduce the likelihood of an incorrect determination of the autoregulation status.

In some examples, processing circuitry 210 presents, via display 220, an indication of the determined autoregulation status of the patient (1310). In some examples, the indication of the autoregulation status may include a color, such as green for intact autoregulation and red for impaired autoregulation. In addition to or instead of the color or other nontextual indication of autoregulation status, the indication of the autoregulation status may also include an indication of the determined limits of autoregulation, the most recent MAP value, the most recent COx value, the COx threshold for the intact region of autoregulation, and so on.

Regional oximetry device 200 and processing circuitry 210 may be configured to determine the relative time delay and synchronize the data sets relatively quickly, as compared to other regional oximetry devices. For example, processing circuitry 210 may determine the relative time delay based on benchtop testing including inputting dummy signals or flicking both of sensors 232 and 234. Processing circuitry 210 may quickly determine a cross-correlation function and the matching time lag over which the cross-correlation function has a maximum amplitude. Processing circuitry 210 may use the durations of the sampling windows, which may be stored to memory 212, to determine the relative time delay. In contrast, another regional oximetry device may use a correlation window over a long period of time, such as five or ten minutes, to reduce the effects of noise or delays, without synchronizing the signals in the manner described in some examples herein.

The relative time delay (e.g., upstream and downstream) between the MAP values and the oxygen saturation values may change over time. Thus, in some examples, processing circuitry 210 may be configured to periodically re-determine the relative time delay during the operation of regional oximetry device 200. For example, processing circuitry 210 may re-determine the relative time delay at regular intervals and/or in response to detection of a detection of a particular event. In some examples, the event is a signal quality metric less than or equal to a threshold value or a signal quality metric that decreases by a certain amount over time. In another example, the event is a change in (e.g., made by processing circuitry 210) to the duration of a sampling window for a physiological signal. In this example, processing circuitry 210 may re-determine the downstream relative time delay.

In some examples, processing circuitry 210 may re-determine the cross-correlation function at regular intervals in order to re-determine the time lag at which the cross-correlation function has a maximum amplitude. In some examples, processing circuitry 210 may change the relative time delay only in response to determining the change in the sampling window is greater than or equal to a threshold level or if in response to determining the change in the time lag of the cross-correlation function is greater than or equal to a threshold level.

FIG. 14 is a flow diagram illustrating an example regional oximetry device 100 including processing circuitry 110 configured to synchronize regional oxygen saturation (rSO₂) and MAP signals. Processing circuitry 110 may be configured to receive the raw NIRS signal from one of sensing devices 132 (1410) and determine a set of oxygen saturation values based on the raw NIRS signal 1110 (1420). MPM device 150 may be configured to receive a raw blood pressure signal from one of sensing devices 182 and determine a set of MAP values based on the raw blood pressure signal. Processing circuitry 110 may receive the set of MAP values from MPM device 150 (1440). In some examples, processing circuitry 110 may receive a blood pressure signal and determine the set of MAP values without receiving a set of MAP values from MPM device 150.

Processing circuitry 110 may determine a reporting delay and the analysis window characteristics for the set of oxygen saturation values (1450). The reporting delay may be based on the hardware for sensing and delivering the NIRS signal to processing circuitry 110. The analysis window characteristics may be based on the determination of the set of oxygen saturation values by processing circuitry 110 (1420). Processing circuitry 110 may determine a reporting delay and the analysis window characteristics for the set of MAP values (1460). The reporting delay may be based on the hardware for sensing and delivering the blood pressure signal to MPM device 150. The reporting delay for the set of MAP values may also be based on the communication interface between processing circuitry 110 and MPM device 150. The analysis window characteristics may be based on the determination of the set of oxygen saturation values by MPM device 150 (1440).

FIG. 14 depicts two types of time delays in the process of determining an autoregulation status. The first type of time delay occurs in receiving of the raw blood pressure and NIRS signals by sensing devices 132 and 182 and the delivery of the raw signals by sensing devices 132 and 182 to processing circuitry 110. The first type of time delay may be referred to as an upstream delay. The second type of time delay occurs in processing the delivered signals by processing circuitry 110. The second type of time delay may result from the durations of the sampling windows or other characteristics of the algorithms used by processing circuitry 110 to determine the set of MAP values and the set of oxygen saturation values. The second type of time delay may be referred to as a downstream delay.

Processing circuitry 110 may synchronize the set of MAP values and the set of oxygen saturation values (1470). Regional oximetry device 100 may determine the relative time delay by subtracting the total delay for the set of MAP values from the total delay for the set of oxygen saturation values. The total delay for each set of values may be the reporting delay plus the sampling window delay (e.g., the first type of time delay plus the second type of time delay). Processing circuitry 110 may shift one set of values by the relative time delay so that the sets are synchronized in time. Processing circuitry 110 may be configured to determine a CA status based on the synchronized sets of values (1480). Processing circuitry 110 may then be configured to present the CA status via display 120 (1490).

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory. The computer-readable storage media may be referred to as non-transitory. A programmer, such as patient programmer or clinician programmer, or other computing device may also contain a more portable removable memory type to enable easy data transfer or offline data analysis.

The techniques described in this disclosure, including those attributed to devices 100, 150, 200, 400, and 500, processing circuitry 110, 160, 210, 410, 414, and 450, memories 112, 162, 212, and 412, controller 214, control circuitry 470, displays 120, 170, 220, 420, and 520, sensing circuitries 130 and 180, sensing devices 132, 182, and 434, and sensors 232 and 234, and/or communication interfaces 140 and 490, and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, remote servers, or other devices. The term “processor” or “processing circuitry” may generally refer to any of the foregoing logic circuitry, alone or in combination with other logic circuitry, or any other equivalent circuitry.

As used herein, the term “circuitry” refers to an ASIC, an electronic circuit, a processor (shared, dedicated, or group) and memory that execute one or more software or firmware programs, a combinational logic circuit, or other suitable components that provide the described functionality. The term “processing circuitry” refers one or more processors distributed across one or more devices. For example, “processing circuitry” can include a single processor or multiple processors on a device. “Processing circuitry” can also include processors on multiple devices, wherein the operations described herein may be distributed across the processors and devices.

Such hardware, software, firmware may be implemented within the same device or within separate devices to support the various operations and functions described in this disclosure. For example, any of the techniques or processes described herein may be performed within one device or at least partially distributed amongst two or more devices, such as between devices 100 and 150, processing circuitry 110 and 160, memories 112 and 162, sensing circuitries 130 and 180, and/or communication interface 140. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in an article of manufacture including a non-transitory computer-readable storage medium encoded with instructions. Instructions embedded or encoded in an article of manufacture including a non-transitory computer-readable storage medium encoded, may cause one or more programmable processors, or other processors, to implement one or more of the techniques described herein, such as when instructions included or encoded in the non-transitory computer-readable storage medium are executed by the one or more processors. Example non-transitory computer-readable storage media may include RAM, ROM, programmable ROM (PROM), erasable programmable ROM (EPROM), electronically erasable programmable ROM (EEPROM), flash memory, a hard disk, a compact disc ROM (CD-ROM), a floppy disk, a cassette, magnetic media, optical media, or any other computer readable storage devices or tangible computer readable media.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache). Elements of devices and circuitry described herein, including, but not limited to, devices 100, 150, 200, 400, and 500, processing circuitry 110, 160, 210, 410, 414, and 450, and/or sensing circuitries 130 and 180 may be programmed with various forms of software. The one or more processors may be implemented at least in part as, or include, one or more executable applications, application modules, libraries, classes, methods, objects, routines, subroutines, firmware, and/or embedded code, for example.

Various examples of the disclosure have been described. Any combination of the described systems, operations, or functions is contemplated. These and other examples are within the scope of the following claims.

What is claimed is:

1. A device comprising:
a display; and
processing circuitry configured to:
receive a physiological signal indicative of a physiological parameter of a patient;
determine a first set of mean arterial pressure values of the patient for a time period based on the physiological signal using a first algorithm;
determine an autoregulation status of the patient based on the first set of mean arterial pressure values; and
present, via the display, a second set of mean arterial pressure values of the patient for the time period, wherein the second set of mean arterial pressure values are determined using a second algorithm different from the first algorithm.

2. The device of claim 1, wherein the processing circuitry is further configured to:
determine the second set of mean arterial pressure values based on the physiological signal using the second algorithm; and
communicate the second set of mean arterial pressure values to a multi-parametric monitor device.

3. The device of claim 1, wherein the processing circuitry is further configured to determine the second set of mean arterial pressure values based on the physiological signal using the second algorithm to mimic determination of mean arterial pressure for the time period by a multi-parametric monitor device.

4. The device of claim 3,
wherein the processing circuitry is configured to determine the second set of values for mean arterial pressure using the second algorithm to mimic determination of mean arterial pressure by at least determining a weighted average of a systolic peak of the physiological signal and a diastolic peak of the physiological signal, and
wherein a weighting of the diastolic peak of the physiological signal is greater than or equal to a weighting of the systolic peak of the physiological signal.

5. The device of claim 1, wherein the processing circuitry is further configured to receive the second set of mean arterial pressure values from a multi-parametric monitor device.

6. The device of claim 1, wherein the physiological parameter of the patient comprises a first physiological parameter, and wherein the processing circuitry is further configured to:
receive a second physiological signal of the patient, the second physiological signal being indicative of a second physiological parameter different from the first physiological parameter;
determine a set of oxygen saturation values of the patient based on the second physiological signal; and
determine a relative time delay between the first set of mean arterial pressure values and the set of oxygen saturation values,
wherein the processing circuitry is configured to determine the autoregulation status of the patient based on the first set of mean arterial pressure values, the set of oxygen saturation values, and the relative time delay between the first set of mean arterial pressure values and the set of oxygen saturation values.

7. The device of claim 6,
wherein the processing circuitry is configured to determine the first set of mean arterial pressure values based on a sampling window for the first set of mean arterial pressure values,
wherein the processing circuitry is configured to determine the set of oxygen saturation values based on a sampling window for the set of oxygen saturation values, and
wherein the processing circuitry is configured to determine the relative time delay between the first set of mean arterial pressure values and the set of oxygen saturation values based on a duration of the sampling window for the first set of mean arterial pressure values and a duration of the sampling window for the set of oxygen saturation values.

8. The device of claim 6, wherein the processing circuitry is configured to determine the relative time delay between the first set of mean arterial pressure values and the set of oxygen saturation values by at least:
determining a cross-correlation function for the first physiological signal and the second physiological signal; and
determining a time lag at which the cross-correlation function has a maximum amplitude.

9. The device of claim 6,
wherein the processing circuitry is further configured to synchronize the first set of mean arterial pressure values and the set of oxygen saturation values based on the relative time delay, and
wherein the processing circuitry is configured to determine the autoregulation status of the patient based on the synchronized first set of mean arterial pressure values and the synchronized set of oxygen saturation values.

10. The device of claim 1, wherein the processing circuitry is further configured to:
determine that the autoregulation status is impaired based on the first set of mean arterial pressure values and a set of oxygen saturation values of the patient, and
generate a notification in response to determining that the autoregulation status is impaired.

11. The device of claim 1, wherein the processing circuitry is further configured to:
determine a signal quality metric based on the difference between the first set of mean arterial pressure values and the second set of mean arterial pressure values,
determine that the signal quality metric is greater than or equal to a threshold level,
generate a notification in response to determining that the signal quality metric is greater than or equal to the threshold level,
output the autoregulation status before determining that the signal quality metric is greater than or equal to the threshold level, and
refrain from further outputting the autoregulation status in response to determining that the signal quality metric is greater than or equal to the threshold level.

12. A method comprising:
receiving, by processing circuitry, a physiological signal indicative of a physiological parameter of a patient;
determining, by the processing circuitry, a first set of mean arterial pressure values of the patient for a time period based on the physiological signal using a first algorithm;
determining, by the processing circuitry, an autoregulation status of the patient based on the first set of mean arterial pressure values; and
presenting, via a display, a second set of mean arterial pressure values of the patient for the time period, wherein the second set of mean arterial pressure values are determined using a second algorithm different from the first algorithm.

13. The method of claim 12, further comprising:
determining the second set of mean arterial pressure values based on the physiological signal using the second algorithm; and
communicating the second set of mean arterial pressure values to a multi-parametric monitor device.

14. The method of claim 12, further comprising receiving the second set of mean arterial pressure values from a multi-parametric monitor device.

15. The method of claim 12, further comprising:
receiving, from the sensing circuitry, a second physiological signal of the patient, the second physiological signal being indicative of a second physiological parameter different from the first physiological parameter;
determining a set of oxygen saturation values of the patient based on the second physiological signal; and
determining a relative time delay between the first set of mean arterial pressure values and the set of oxygen saturation values,
wherein determining the autoregulation status of the patient is based on the first set of mean arterial pressure values, the set of oxygen saturation values, and the relative time delay between the first set of mean arterial pressure values and the set of oxygen saturation values.

16. The method of claim 15,
wherein determining the first set of mean arterial pressure values is further based on a sampling window for the first set of mean arterial pressure values,
wherein determining the set of oxygen saturation values is further based on a sampling window for the set of oxygen saturation values,
wherein determining the relative time delay between the first set of mean arterial pressure values and the set of oxygen saturation values comprises determining the relative time delay based on a duration of the sampling window for the first physiological signal and a duration of the sampling window for the second physiological signal.

17. The method of claim 15, wherein determining the relative time delay between the first set of mean arterial pressure values and the set of oxygen saturation values comprises:
determining a cross-correlation function for the first physiological signal and the second physiological signal; and
determining a time lag at which the cross-correlation function has a maximum amplitude.

18. A device comprising:
a display; and
processing circuitry configured to:
receive a first physiological signal indicative of a physiological parameter of a patient;
determine a first set of mean arterial pressure values of the patient for a time period based on the first physiological signal;
transmit the first set of mean arterial pressure values to a multi-parametric monitor device;
determine an autoregulation status of the patient based on the first set of mean arterial pressure values; and
present, via the display, the first set of mean arterial pressure values of the patient for the time period.

19. The device of claim 18, wherein the physiological parameter of the patient comprises a first physiological parameter, and wherein the processing circuitry is further configured to:
receive a second physiological signal of the patient, the second physiological signal being indicative of a second physiological parameter different from the first physiological parameter;
determine a set of oxygen saturation values of the patient based on the second physiological signal; and
determine a relative time delay between the first set of mean arterial pressure values and the set of oxygen saturation values,
wherein the processing circuitry is configured to determine the autoregulation status of the patient based on the first set of mean arterial pressure values, the set of oxygen saturation values, and the relative time delay between the first set of mean arterial pressure values and the set of oxygen saturation values.

20. The device of claim 18,
wherein the processing circuitry is configured to determine the first set of mean arterial pressure values based on a sampling window for the first set of mean arterial pressure values,
wherein the processing circuitry is configured to determine the set of oxygen saturation values based on a sampling window for the set of oxygen saturation values, and
wherein the processing circuitry is configured to determine the relative time delay between the first set of mean arterial pressure values and the set of oxygen saturation values based on a duration of the sampling window for the first set of mean arterial pressure values and a duration of the sampling window for the set of oxygen saturation values.
